SEQUENCE LISTING (1 sequence)

SEQ ID NO: 1 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2009/0203601 A1
(43) Pub. Date: Aug. 13, 2009

(54) Method for the treatment of neutropenia by administration of a multi-pegylated granulocyte colony stimulating factor (G-CSF) variant

(75) Inventors: Bobby Soni, Kongens Lyngby (DK); Torben Straight Nissen, Summit, NJ (US); Mads Roepke, Hellerup (DK); David Gilfoyle, Oakham (GB)

Correspondence Address: MAXYGEN, INC.;INTELLECTUAL PROPERTY DEPARTMENT, 515 GALVESTON DRIVE, REDWOOD CITY, CA, 94063, US

(73) Assignee: Maxygen Holdings Ltd.

(21) Appl. No.: 12/313,902

(22) Filed: Nov. 25, 2008

Related U.S. Application Data
(63) Continuation-in-part of application No. PCT/US2008/006618, filed on May 22, 2008.
(60) Provisional application No. 60/939,524, filed on May 22, 2007.

(51) Int. Cl. A61K 38/18; A61P 35/00

(52) U.S. Cl. 514/12

(57) ABSTRACT

The invention relates to a method for treating or preventing neutropenia in a patient receiving chemotherapy by administering a multi-PEGylated granulocyte colony stimulating factor (G-CSF) variant on the same day that chemotherapy is administered.

### CROSS-REFERENCE TO RELATED APPLICATIONS

This continuation in part application claims priority, pursuant U.S.C. § 120, to International application No. PCT/US2008/006618, filed May 22, 2008, which claims the benefit of U.S. Ser. No. 60/939,524, filed May 22, 2007, under 35 U.S.C. § 119(e), both of which applications are incorporated herein by reference in their entireties.

### FIELD OF THE INVENTION

The present invention relates to a method for treating or preventing neutropenia by administering a multi-PEGylated granulocyte colony stimulating factor (G-CSF) variant.

### BACKGROUND OF THE INVENTION

The process by which white blood cells grow, divide and differentiate in the bone marrow is called hematopoiesis (Dexter and Spooncer, Ann. Rev. Cell. Biol., 3:423, 1987). Each of the blood cell types arises from pluripotent stem cells. There are generally three classes of blood cells produced in vivo: red blood cells (erythrocytes), platelets and white blood cells (leukocytes), the majority of the latter being involved in host immune defense. Proliferation and differentiation of hematopoietic precursor cells are regulated by a family of cytokines, including colony-stimulating factors (CSF's) such as G-CSF and interleukins (Arai et al., Ann. Rev. Biochem., 59:783-836, 1990). The principal biological effect of G-CSF in vivo is to stimulate the growth and development of certain white blood cells known as neutrophilic granulocytes or neutrophils (Welte et al., PNAS-USA 82:1526-1530, 1985, Souza et al., Science, 232:61-65, 1986). When released into the blood stream, neutrophilic granulocytes function to fight bacterial and other infections.

The amino acid sequence of human G-CSF (hG-CSF) was reported by Nagata et al. Nature 319:415-418, 1986. hG-CSF is a monomeric protein that dimerizes the G-CSF receptor by formation of a 2:2 complex of 2 G-CSF molecules and 2 receptors (Horan et al. (1996), Biochemistry 35(15): 4886-96). In a more recent publication (PNAS, Feb. 28, 2006, vol. 103, No. 9:3135-3140), Tamada et al. describe a crystal structure of the signaling complex between human G-CSF and a ligand binding region of the GCSF receptor.

Leukopenia (a reduced level of white blood cells) and neutropenia (a reduced level of neutrophils) are disorders that result in an increased susceptibility to various types of infections. Neutropenia can be chronic, e.g. in patients infected with HIV, or acute, e.g. in cancer patients undergoing chemotherapy or radiation therapy. For patients with severe neutropenia, exhibited by an absolute neutrophil count (ANC) below about 500 cells/mm³, even relatively minor infections can be serious and even life-threatening. Recombinant human G-CSF (rhG-CSF) is used for treating and preventing various forms of leukopenia and neutropenia, the general aim being to maintain the optimal chemotherapy dose and avoid having to reduce the dose or delay administration of chemotherapy as a result of neutropenia. Preparations of rhG-CSF are commercially available, e.g. Neupogen® (Filgrastim), which is non-glycosylated and produced in recombinant E. coli cells, and Neulasta® (Pegfilgrastim), which is the same as Neupogen® but contains a single N-terminally linked 20 kDa polyethylene glycol (PEG) group. This PEGylated G-CSF molecule has been shown to have an increased half-life compared to non-PEGylated G-CSF and thus may be administered less frequently than the non-PEGylated G-CSF products, and it reduces the duration of neutropenia to about the same number of days as non-PEGylated G-CSF.

Although Neulasta® has the advantage that it can be administered less frequently than non-PEGylated G-CSF such as Neupogen®, e.g. once every cycle of chemotherapy rather than once a day, it nevertheless suffers from the disadvantage that it is not approved for administration simultaneously with or on the same day as chemotherapy (or, in situations in which an administration cycle of chemotherapy is conducted for more than one day, on the last day of such a regimen). In particular, the product information for Neulasta® specifies that it should not be administered in the period between 14 days before and 24 hours after administration of cytotoxic chemotherapy because of the potential for an increase in sensitivity of rapidly dividing myeloid cells to cytotoxic chemotherapy (Neulasta® prescribing information, Amgen, publication date Sep. 15, 2005). The requirement for next-day administration of G-CSF represents a significant disadvantage for chemotherapy patients, who must return to the hospital on the day following chemotherapy in order to receive their G-CSF treatment. If G-CSF could instead be administered on the same day as chemotherapy, this would provide an important convenience advantage not only to patients, but also a cost savings to hospitals and health care professionals.

A number of studies relating to same-day versus next-day administration of filgrastim or PEG-filgrastim have been reported. However, the results of these studies are ambiguous, and the non-conclusive nature of the experimental results currently available is compounded by the fact that results may vary depending on factors such as the particular nature of the cancer being treated and the type of chemotherapy.

For filgrastim, some studies have concluded that same-day administration of G-CSF and cytotoxic chemotherapy results in severe myelosuppression (e.g. Meropol et al. (1992), J. Nat. Cancer Inst. 84(15):1201-3; Rowinsky et al. (1996), J. Clin. Oncol. 14:1224-1235; see also the reviews by Petros et al. (1997), Current Opinion in Hematology 4:213-216, and Rowe et al. (2000), Current Opinion in Hematology 7:197-202), while other studies have concluded that concurrent administration of G-CSF and chemotherapy may be feasible (e.g. Livingston et al. (1997), J. Clin. Oncol. 15:1395-1400; Ellis et al. (1998), ASCO 1998, Abst. No. 528; Ottmann et al. (1995), Blood 86(2):444-450).

A similar picture is seen for PEG-filgrastim, with some reports suggesting that same-day administration of PEG-filgrastim and chemotherapy leads to more severe myelosuppression (e.g. Kaufman (2004), San Antonio Breast Cancer Symposium 2004, 88 (suppl. 1):S59, Abst. No. 1054; Yardley et al. (2005), ASCO 2005, Abst. No. 749), while others suggest that simultaneous administration of PEG-filgrastim and chemotherapy may be safe in some situations (e.g. Lokich (2005), Cancer Investigation 23:573-576; Hoffmann (2005), ASCO 2005, Abst. No. 8137; Watt et al. (2004), Blood (ASH Ann. Meeting Abstracts), Abst. No. 2215).

For PEG-filgrastim in particular, the data is relatively new, and although some researchers have concluded that G-CSF may be administered on the same day as chemotherapy in some situations, other studies have concluded the opposite. Thus, there is substantial uncertainty as to whether Neulasta®, the only commercially available PEG-filgrastim product, could be administered safely in a same-day protocol for any chemotherapy regimen.

There is therefore a need for long-acting G-CSF products, in particular PEGylated G-CSF, that can safely be administered on the same day as chemotherapy, and for methods for treatment and prevention of chemotherapy-induced neutropenia using such G-CSF products.

### BRIEF DESCRIPTION OF THE INVENTION

The object of the present invention is to provide a method of treatment that allows G-CSF to be administered to a patient on the same day that the patient receives chemotherapy.

One aspect of the invention thus relates to a method for treating or preventing neutropenia in a patient receiving chemotherapy, comprising administering to said patient a multi-PEGylated G-CSF variant in an amount effective to reduce chemotherapy-induced neutropenia, wherein said PEGylated G-CSF is administered to the patient on the same day as chemotherapy.

A further aspect of the invention relates to a multi-PEGylated G-CSF variant for treating or preventing neutropenia by means of the method described herein. This aspect of the invention thus relates to a multi-PEGylated G-CSF variant for the same day treatment of chemotherapy-induced neutropenia. This aspect of the invention also relates to a multi-PEGylated G-CSF variant for treating or preventing neutropenia in a patient receiving chemotherapy by administering the multi-PEGylated G-CSF variant to the patient on the same day that the patient receives chemotherapy.

A further aspect of the invention relates to use of a multi-PEGylated G-CSF variant for the preparation of a medicament for treating or preventing neutropenia by means of the method described herein. This aspect of the invention thus relates to use of a multi-PEGylated G-CSF variant for the preparation of a medicament for treating or preventing neutropenia in a patient receiving chemotherapy, wherein the multi-PEGylated G-CSF variant is administered to the patient in an amount effective to reduce chemotherapy-induced neutropenia, and wherein said multi-PEGylated G-CSF variant is administered to the patient on the same day as chemotherapy. This aspect of the invention also relates to use of a multi PEGylated G-CSF variant for the preparation of a medicament for the same day treatment of chemotherapy-induced neutropenia. This aspect of the invention also relates to use of a multi-PEGylated G-CSF variant for the preparation of a medicament for treating or preventing neutropenia in a patient receiving chemotherapy by administering the multi-PEGylated G-CSF variant to the patient on the same day that the patient receives chemotherapy.

### BRIEF DESCRIPTION OF THE DRAWINGS

FIG. 1A shows the effect of different doses of Maxy-G on leukocyte cell counts in rats pretreated with Cyclophosphamide (CPA). Maxy-G was administered two hours after the CPA.

FIG. 1B shows the effect of different doses of Neulasta® on leukocyte cell counts on CPA-induced leukopenia in rats. Neulasta® was administered two hours after the CPA.

FIG. 1C shows the comparative effect of equivalent doses (100 μg/kg) of Maxy-G and Neulasta® on CPA-induced leukopenia in rats. Both Maxy-G and Neulasta® were administered two hours after the CPA.

FIG. 1D shows the comparative effect of equivalent doses (300 μg/kg) of Maxy-G and Neulasta® on CPA-induced leukopenia in rats. Both Maxy-G and Neulasta® were administered two hours after the CPA.

FIG. 2A shows the effect of different doses of Maxy-G on neutrophil cell counts in rats pretreated with CPA. Maxy-G was administered two hours after the CPA.

FIG. 2B shows the effect of different doses of Neulasta® on neutrophil cell counts in rats pretreated with CPA. Neulasta® was administered two hours after the CPA.

FIG. 2C shows the comparative effect of equivalent doses (100 μg/kg) of Maxy-G and Neulasta® on CPA-induced neutropenia in rats. Both Maxy-G and Neulasta® were administered two hours after the CPA.

FIG. 2D shows the comparative effect of equivalent doses (300 μg/kg) of Maxy-G and Neulasta® on CPA-induced neutropenia in rats. Both Maxy-G and Neulasta® were administered two hours after the CPA.

FIG. 3 shows the effect of various doses of Maxy-G and Neulasta® on Paclitaxel (Taxol)-induced neutropenia in rats. Vehicle (♦), Maxy-G at 0.1 mg/kg (▪) or 0.3 mg/kg (▴), or Neulasta® at 0.1 mg/kg (X) or 0.3 mg/kg ( [symbol] ) were administered two hours after Taxol. The two horizontal lines indicate approximate levels for low normal and high normal neutrophil counts.

FIG. 4 compares the effect of Maxy-G on Doxorubicin-induced neutropenia in rats when Maxy-G is administered either the same day as or the day after Doxorubicin treatment. On day 0, rats were intravenously administered saline (♦) or 4 mg/kg Doxorubicin (▪, ▴, X), followed by a subcutaneous injection of either: vehicle 2 hours after Doxorubicin (♦, ▪); 0.3 mg/kg Maxy-G two hours after Doxorubicin (▴); or 0.3 mg/kg Maxy-G twenty-four hours after Doxorubicin (X). The two horizontal lines indicate approximate levels of low normal and high normal neutrophil counts.

FIG. 5 compares the effect of Maxy-G on Carboplatin-induced neutropenia in rats when Maxy-G is administered either the same day as or the day after Carboplatin treatment. On day 0, rats were intravenously administered saline (♦) or 40 mg/kg Carboplatin (▪, ▴, X), followed by a subcutaneous injection of either: vehicle 2 hours after Carboplatin (♦, ▪); 0.3 mg/kg Maxy-G two hours after Carboplatin (▴); or 0.3 mg/kg Maxy-G twenty-four hours after Carboplatin (X). The two horizontal lines indicate approximate levels of low normal and high normal neutrophil counts.

FIG. 6 compares the effect of Maxy-G on Cyclophosphamide-induced neutropenia in rats when Maxy-G is administered either the same day as or the day after Cyclophosphamide treatment. On day 0, rats were intravenously administered saline (♦) or 20 mg/kg Cyclophosphamide (▪, ▴, X), followed by a subcutaneous injection of either: vehicle 2 hours after Cyclophosphamide (♦, ▪); 0.3 mg/kg Maxy-G two hours after Cyclophosphamide (▴); or 0.3 mg/kg Maxy-G twenty-four hours after Cyclophosphamide (X). The two horizontal lines indicate approximate levels of low normal and high normal neutrophil counts.

FIG. 7 compares the effect of Maxy-G on Vincristine-induced neutropenia in rats when Maxy-G is administered either the same day as or the day after Vincristine treatment. On day 0, rats were intravenously administered saline (♦) or 0.15 mg/kg body weight Vincristine (▪, ▴, X), followed by a subcutaneous injection of either: vehicle 2 hours after Vincristine (♦, ▪); 0.3 mg/kg Maxy-G two hours after Vincristine (▴); or 0.3 mg/kg Maxy-G twenty-four hours after Vincristine (X). The two horizontal lines indicate approximate levels of low normal and high normal neutrophil counts.

FIG. 8A compares the effect of 200 μg/kg Maxy-G to 200 μg/kg Neulasta® on leukocyte cell counts in rats pretreated with Cyclophosphamide (CPA) when Maxy-G (, ▪ and ▾) and Neulasta® (◯, □ and ∇) were each administered one-half hour, 2 hours or 24 hours after the CPA.

FIG. 8B compares the effect of 200 μg/kg Maxy-G to 200 μg/kg Neulasta® on absolute neutrophil counts in rats pretreated with CPA when Maxy-G (, ▪ and ▾) and Neulasta® (◯, □ and ∇) were each administered one-half hour, 2 hours or 24 hours after the CPA.

FIG. 8C shows the effect of Maxy-G34 on absolute neutrophil counts in rats pretreated with CPA when Neulasta® was administered at one-half hour (), 2 hours (▪) or 24 hours (▾) after the CPA.

FIG. 8D shows the effect of Neulasta® on absolute neutrophil counts in rats pretreated with CPA when Maxy-G34 was administered at one-half hour (◯), 2 hours (□) or 24 hours (∇) after the CPA

FIG. 8E compares the effect of 200 μg/kg Maxy-G to 200 μg/kg Neulasta® on absolute neutrophil counts in rats pretreated with CPA when Maxy-G () and Neulasta® (◯) were each administered one-half hour after the CPA.

FIG. 8F compares the effect of 200 μg/kg Maxy-G to 200 μg/kg Neulasta® on absolute neutrophil counts in rats pretreated with CPA when Maxy-G (▪) and Neulasta® (□) were each administered 2 hours after the CPA.

FIG. 8G compares the effect of 200 μg/kg Maxy-G to 200 μg/kg Neulasta® on absolute neutrophil counts in rats pretreated with CPA when Maxy-G (▾) and Neulasta® (∇) were each administered 24 hours after the CPA.

FIG. 9A compares the effect of 100 μg/kg Maxy-G to 100 μg/kg Neulasta® on leukocyte cell counts in rats pretreated with Cyclophosphamide (CPA) when Maxy-G (, ▾ and ♦) and Neulasta® (◯, ∇ and ⋄) were each administered one-half hour, 24 hours or 48 hours after the CPA.

FIG. 9B compares the effect of 100 μg/kg Maxy-G to 100 μg/kg Neulasta® on absolute neutrophil counts in rats pretreated with CPA when Maxy-G (, ▾ and ♦) and Neulasta® (◯, ∇ and ⋄) were each administered one-half hour, 24 hours or 48 hours after the CPA.

### DEFINITIONS

In the description and claims below, the follow definitions apply.

The terms “polypeptide” or “protein” may be used interchangeably herein to refer to polymers of amino acids, without being limited to an amino acid sequence of any particular length. These terms are intended to include not only full-length proteins but also e.g. fragments or truncated versions, variants, domains, etc. of any given protein or polypeptide.

A “G-CSF polypeptide” is a polypeptide having the sequence of human granulocyte colony stimulating factor (hG-CSF) as shown in SEQ ID NO:1, or a variant of hG-CSF that exhibits G-CSF activity. The “G-CSF activity” may be the ability to bind to a G-CSF receptor (Fukunaga et al., J. Bio. Chem, 265:14008, 1990, which is incorporated herein by reference), but is preferably G-CSF cell proliferation activity, in particular determined in an in vitro activity assay using the murine cell line NFS-60 (ATCC Number: CRL-1838). A suitable in vitro assay for G-CSF activity using the NFS-60 cell line is described by Hammerling et al. in J. Pharm. Biomed. Anal. 13(1):9-20, 1995, which is incorporated herein by reference. A polypeptide “exhibiting” G-CSF activity is considered to have such activity when it displays a measurable function, e.g. a measurable proliferative activity in the in vitro assay.

A “variant” (e.g., a “G-CSF variant”) is a polypeptide which differs in one or more amino acid residues from a parent polypeptide, where the parent polypeptide is generally one with a native, wild-type amino acid sequence, typically a native mammalian polypeptide, and more typically a native human polypeptide. The variant thus contains one or more substitutions, insertions or deletions compared to the native polypeptide. These may, for example, include truncation of the N- and/or C-terminus by one or more amino acid residues, or addition of one or more extra residues at the N- and/or C-terminus, e.g. addition of a methionine residue at the N-terminus. The variant will most often differ in up to 15 amino acid residues from the parent polypeptide, such as in up to 12, 10, 8 or 6 amino acid residues. Some G-CSF variants, in particular, have amino acid substitutions in the G-CSF sequence either with or without the addition of a methionine residue at the N-terminus.

The term “modified” G-CSF refers to a G-CSF molecule with either the sequence of human G-CSF or a variant of human G-CSF, that is modified by, e.g., alteration of the glycan structure. For example, the glycan structure of G-CSF may be modified for the purpose of providing glyco-PEGylated G-CSF molecules in which polyethylene glycol moieties are attached to a glycosyl linking group such as a sialic acid moiety as described in WO 2005/055946. Another example of a modified G-CSF molecule is one that contains at least one O-linked glycosylation site that does not exist in the wild-type polypeptide. G-CSF molecules having such non-naturally occurring O-linked glycosylation sites, as well as PEGylation of modified sugars of G-CSF, are described in WO 2005/070138, which is incorporated herein by reference.

Unless otherwise indicated, the term “G-CSF” as used herein is intended to encompass G-CSF molecules with the native human sequence (SEQ ID NO:1) as well as variants of the human G-CSF sequence. In either case, the term “G-CSF” is also intended to include modified G-CSF such as G-CSF glycosylation variants.

A PEGylated G-CSF that “comprises multiple polyethylene glycol moieties” (also referred to herein as a “multi-PEGylated G-CSF”) refers to a G-CSF polypeptide having two or more PEG moieties that are covalently attached either directly or indirectly to an amino acid residue of the polypeptide. Suitable attachment sites include, for example, the ε-amino group of a lysine residue or the N-terminal amino group, a free carboxylic acid group (e.g. that of the C-terminal amino acid residue or of an aspartic acid or glutamic acid residue), the thiol group of a cysteine residue, suitably activated carbonyl groups, oxidized carbohydrate moieties and mercapto groups. More information on PEG attachment sites and methods for attachment of PEG moieties to proteins may be found, e.g., in WO 01/51510, WO 03/006501, and the Nektar Advanced PEGylation Catalog 2005-2006 (Nektar Therapeutics), all of which are incorporated herein by reference. Another possibility for PEGylation is to attach PEG moieties to the glycan structures of G-CSF, e.g. by way of glycan modification (see above).

A “multi-PEGylated G-CSF variant” refers to a G-CSF variant having two or more PEG moieties that are covalently attached either directly or indirectly to an amino acid residue of the variant.

In the present application, amino acid names and atom names (e.g. CA, CB, NZ, N, O, C, etc.) are used as defined by the Protein DataBank (PDB), which is based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985). The term “amino acid residue” is intended to indicate any naturally or non-naturally occurring amino acid residue, in particular an amino acid residue contained in the group consisting of the 20 naturally occurring amino acids, i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

The terminology used for identifying amino acid positions/substitutions herein is illustrated as follows: F13 indicates position number 13 occupied by a phenylalanine residue in the reference amino acid sequence. F13K indicates that the phenylalanine residue of position 13 has been substituted with a lysine residue. Unless otherwise indicated, the numbering of amino acid residues made herein is made relative to the amino acid sequence of hG-CSF shown in SEQ ID NO:1. Alternative substitutions are indicated with a “/”, e.g. K16R/Q means an amino acid sequence in which lysine in position 16 is substituted with either arginine or glutamine. Multiple substitutions are indicated with a “+”, e.g. K40R+T105K means an amino acid sequence which comprises a substitution of the lysine residue in position 40 with an arginine residue and a substitution of the threonine residue in position 105 with a lysine residue.

The term “functional in vivo half-life” is used in its normal meaning, i.e. the time at which 50% of the biological activity of the test molecule (e.g., PEGylated conjugate) is still present in the body/target organ, or the time at which the activity of the polypeptide or conjugate is 50% of the initial value. “Serum half-life” is defined as the time in which 50% of the conjugate molecules circulate in the plasma or bloodstream prior to being cleared. Alternative terms to serum half-life include “plasma half-life”, “circulating half-life”, “serum clearance”, “plasma clearance” and “clearance half-life”. The test molecule (e.g., PEGylated conjugate) is cleared by the action of one or more of the reticuloendothelial systems (RES), kidney, spleen or liver, by receptor-mediated degradation, or by specific or non-specific proteolysis, in particular by the action of receptor-mediated clearance and renal clearance. Normally, clearance depends on size (relative to the cutoff for glomerular filtration), charge, attached carbohydrate chains, and the presence of cellular receptors for the protein. The functionality to be retained is normally selected from proliferative or receptor-binding activity. The functional in vivo half-life and the serum half-life may be determined by any suitable method known in the art or as described in the Materials and Methods section below.

The term “increased” as used in reference to in vivo half-life or serum half-life is used to indicate that the half-life of the test molecule, i.e. the multi-PEGylated G-CSF variant, is statistically significantly increased relative to that of a reference molecule, such as a non-conjugated (i.e., non-PEGylated) hG-CSF (e.g. Neupogen®) or preferably, relative to the mono-PEGylated G-CSF Neulasta®, as determined under comparable conditions (typically determined in an experimental animal, such as rat, rabbit, pig or monkey). For instance, the serum half-life (t_{1/2}) of the test molecule may be increased by at least about 1.2× to that of the reference molecule (that is, (t_{1/2} of the test molecule)/(t_{1/2} of the reference molecule)=1.2), e.g. by at least about 1.4×, such as by at least about 1.5×, e.g. by at least about 1.6×, such as by at least about 1.8×, e.g. by at least about 2.0×, 2.5×, 3×, 5×, or 10× to that of the reference molecule.

The term “AUC” or “Area Under the Curve” is used in its normal meaning, i.e. as the area under the serum concentration versus time curve where the test molecule has been administered to a subject. Once the experimental concentration-time points have been determined, the AUC may conveniently be calculated by a computer program, such as GraphPad Prism 3.01.

The term “increased” as used in reference to the AUC is used to indicate that the AUC of the test molecule, i.e. the multi-PEGylated G-CSF variant, is statistically significantly increased relative to that of a reference molecule, such as a non-conjugated hG-CSF (e.g. Neupogen®) or preferably, relative to the mono-PEGylated G-CSF Neulasta®, as determined under comparable conditions (typically determined in an experimental animal, such as rat, rabbit, pig or monkey). For instance, the AUC of the test molecule may be increased by at least about 1.2× to that of the reference molecule (that is, (AUC of the test molecule)/(AUC of the reference molecule)=1.2), e.g. by at least about 1.4×, such as by at least about 1.5×, e.g. by at least about 1.6×, such as by at least about 1.8×, e.g. by at least about 2.0×, 2.5×, 3×, 5×, or 10× to that of the reference molecule.

The term “same-day” or “same day”, for example, “same-day administration” or “administration on the same day as chemotherapy” refers to the fact that according to the invention a multi-PEGylated G-CSF variant is administered to a patient on the same day that the patient receives chemotherapy, i.e. within about 12 hours from the completion of chemotherapy, typically within about 10 hours, more typically within about 8 hours, still more typically within about 6 hours from the completion of chemotherapy. Preferably, the multi-PEGylated G-CSF variant is administered to the patient within about 5 hours from the completion of chemotherapy, more preferably within about 4 hours from the completion of chemotherapy, such as within about 3 hours or within about 2 hours from the completion of chemotherapy. Same-day administration can thus include administration within less than 2 hours from the completion of chemotherapy, such as for example, within about a half hour, within about an hour, or within about an hour and a half from the completion of chemotherapy. It will be understood that for chemotherapy regimens in which administration of the chemotherapy is carried out over more than one day (i.e., a “multi-day regimen”), same-day is in reference to the last day the patient receives chemotherapy, such that the multi-PEGylated G-CSF variant is administered on the same day as the completion of chemotherapy in the multi-day regimen.

### DETAILED DESCRIPTION OF THE INVENTION

The present invention provides a method for treating or preventing neutropenia in a patient receiving chemotherapy, where the method comprises administering to said patient a multi-PEGylated G-CSF variant in an amount effective to reduce chemotherapy-induced neutropenia, wherein the multi-PEGylated G-CSF variant is administered to the patient on the same day as chemotherapy.

We have discovered that administration of a multi-PEGylated G-CSF variant on the same day as chemotherapy is actually significantly more effective at reducing the duration of chemotherapy-induced neutropenia when compared to both a control and mono-PEGylated hG-CSF (Neulasta®) in a chemotherapy rat model. The reduction of time to absolute neutrophil recovery (ANC) was also significantly improved as compared to both the control and mono-PEGylated hG-CSF (Neulasta®). As used herein, term “time to ANC recovery” is defined as the number of days starting from day one of chemotherapy until the first of two consecutive days where the subject has counts above 0.5×10⁹ ANC cells/L, i.e., above the defining limit for severe neutropenia. Time to ANC recovery, duration/days of leukopenia, and duration/days of severe neutropenia are all indicative of the period during which a patient undergoing chemotherapy is in an immune suppressed state (the terms “days of neutropenia” and “days of severe neutropenia” are used interchangeably herein). During this period, the patient is vulnerable to infections which may disrupt the timing of the next cycle of chemotherapy or which may even lead to mortality. In view of the results described in the examples herein, it is contemplated that the administration of the multi-PEGylated G-CSF variant is as effective when administered the same day as chemotherapy as when it is administered the day after chemotherapy, i.e., “next day” administration.

The method of the invention is effective at reducing the time to ANC recovery, days of leukopenia, and days of neutropenia. At equivalent doses, the method is more effective at reducing the time to ANC recovery, days of leukopenia, and days of neutropenia when compared to mono-PEGylated hG-CSF (Neulasta®). In accordance with the method of the present invention, the multi-PEGylated G-CSF variant is administered within the same day as the last day that the patient receives chemotherapy. For example, it may be administered within about 0.5 (i.e., one-half), about 1, about 1.5, about 2, about 3, about 4, about 5, about 6, about 8, about 10, or about 12 hours from the completion of chemotherapy in a given cycle.

Depending on the prognosis of the patient, chemotherapy may be administered multiple cycles over the course of a treatment regimen. Because the multi-PEGylated G-CSF variant is effective at reducing the time to ANC recovery, the duration/days of leukopenia, and the duration/days of neutropenia such that the duration of exposure to risk of infection is lessened, it is contemplated that the multi-PEGylated G-CSF variant may be administered on the same day of the completion of chemotherapy during one or more further cycles of chemotherapy, i.e., without disruption to the timing of chemotherapy cycles in the prescribed treatment regimen. Depending on the chemotherapy agent(s), each cycle may last from about 7 days (1 week) to about 28 days (4 weeks). It is contemplated that the multi-PEGylated G-CSF variant would be administered on the same day as the last day of chemotherapy in one or more chemotherapy cycles of 7 days, 14 days, 21 days, or 28 days, for two, three, four, five, or six or more consecutive cycles of chemotherapy. As used herein, the term “cycle” refers to the period between the first days of administration of chemotherapy in two consecutive cycles of chemotherapy.

### Multi-PEGylated G-CSF Variant

Multi-pegylated proteins may be prepared in a number of ways that are well known in the art. The covalent attachment (i.e., conjugation) of polyethylene glycol (PEG) moieties to proteins or polypeptides (“PEGylation”) is a well-known technique for improving the properties of such proteins or polypeptides, in particular pharmaceutical proteins, e.g. in order to improve circulation half-life and/or to shield potential epitopes and thus reduce the potential for an undesired immunogenic response. Numerous technologies based on activated PEG are available to provide attachment of the PEG moiety to one or more groups on the protein. For example, mPEG-succinimidyl propionate (mPEG-SPA, available from Nektar Therapeutics) is generally regarded as being selective for attachment to the N-terminus and ε-amino groups of lysine residues via an amide bond. As noted above, the commercially available PEGylated G-CSF product Neulasta® contains a single 20 kDa PEG moiety at the N-terminus of the G-CSF molecule.

In some embodiments, multi-PEGylated G-CSF variants described herein exhibit improved pharmacokinetic parameters, such as an increased serum half-life and/or and an increased area under the curve (AUC), relative to the mono-PEGylated G-CSF Neulasta® (pegfilgrastim) when tested in experimental animals such as rats. In accordance with the present invention, a multi-PEGylated G-CSF variant has been found to be advantageous over the mono-PEGylated G-CSF Neulasta® in an animal model of same-day administration when tested with different chemotherapeutic agents, providing a shorter time-to-recovery and a shorter period of neutropenia/leukopenia at equivalent doses.

In one embodiment, the multi-PEGylated G-CSF variant administered according to the invention may be PEGylated with an amine-specific activated PEG that preferentially attaches to the N-terminal amino group and/or to the ε-amino groups of lysine residues via an amide bond. Examples of amine-specific activated PEG derivatives include mPEG-succinimidyl propionate (mPEG-SPA), mPEG-succinimidyl butanoate (mPEG-SBA) and mPEG-succinimidyl α-methylbutanoate (mPEG-SMB) (available from Nektar Therapeutics; see the Nektar Advanced PEGylation Catalog 2005-2006, “Polyethylene Glycol and Derivatives for Advanced PEGylation”); PEG-SS (Succinimidyl Succinate), PEG-SG (Succinimidyl Glutarate), PEG-NPC (p-nitrophenyl carbonate), and PEG-isocyanate, available from SunBio Corporation; and PEG-SCM, available from NOF Corporation. The polyethylene glycol may be either linear or branched.

Methods for obtaining PEGylated proteins are well known in the art; see e.g. the Nektar Advanced PEGylation Catalog 2005-2006, which is incorporated herein by reference. PEGylated G-CSF variants, and methods for their preparation, are e.g. described in WO 01/51510, WO 03/006501, U.S. Pat. No. 6,646,110, U.S. Pat. No. 6,555,660 and U.S. Pat. No. 6,831,158, all of which are incorporated herein by reference.

In a preferred embodiment, the multi-PEGylated G-CSF variant comprises a PEG moiety attached to the N-terminus and at least one PEG moiety attached to a lysine residue.

In one embodiment, the administered multi-PEGylated G-CSF variant comprises at least one substitution in the hG-CSF sequence of SEQ ID NO: 1 to introduce a lysine residue in a position where PEGylation is desired. In particular, the lysine residue may be introduced by way of one or more substitutions selected from the group consisting of T1K, P2K, L3K, G4K, P5K, A6K, S7K, S8K, L9K, P10K, Q11K, S12K, F13K, L14K, L15K, E19K, Q20K, V21K, Q25K, G26K, D27K, A29K, A30K, E33K, A37K, T38K, Y39K, L41K, H43K, P44K, E45K, E46K, V48K, L49K, L50K, H52K, S53K, L54K, I56K, P57K, P60K, L61K, S62K, S63K, P65K, S66K, Q67K, A68K, L69K, Q70K, L71K, A72K, G73K, S76K, Q77K, L78K, S80K, F83K, Q86K, G87K, Q90K, E93K, G94K, S96K, P97K, E98K, L99K, G100K, P101K, T102K, D104K, T105K, Q107K, L108K, D109K, A111K, D112K, F113K, T115K, T116K, W118K, Q119K, Q120K, M121K, E122K, E123K, L124K, M126K, A127K, P128K, A129K, L130K, Q131K, P132K, T133K, Q134K, G135K, A136K, M137K, P138K, A139K, A141K, S142K, A143K, F144K, Q145K, S155K, H156K, Q158K, S159K, L161K, E162K, V163K, S164K, Y165K, V167K, L168K, H170K, L171K, A172K, Q173K and P174K (where residue position is relative to SEQ ID NO: 1).

Examples of preferred amino acid substitutions thus include one or more of Q70K, Q90K, T105K, Q120K, T133K, S159K and H170K/Q/R, such as two, three, four or five of these substitutions, for example: Q70K+Q90K, Q70K+T105K, Q70K+Q120K, Q70K+T133K, Q70K+S159K, Q70K+H170K, Q90K+T105K, Q90K+Q120K, Q90K+T133K, Q90K+S159K, Q90K+H170K, T105K+Q120K, T105K+T133K, T105K+S159K, T105K+H170K, Q120K+T133K, Q120K+S159K, Q120K+H170K, T133K+S159K, T133K+H170K, S159K+H170K, Q70K+Q90K+T105K, Q70K+Q90K+Q120K, Q70K+Q90K+T133K, Q70K+Q90K+S159K, Q70K+Q90K+H170K, Q70K+T105K+Q120K, Q70K+T105K+T133K, Q70K+T105K+S159K, Q70K+T105K+H170K, Q70K+Q120K+T133K, Q70K+Q120K+S159K, Q70K+Q120K+H170K, Q70K+T133K+S159K, Q70K+T133K+H170K, Q70K+S159K+H170K, Q90K+T105K+Q120K, Q90K+T105K+T133K, Q90K+T105K+S159K, Q90K+T105K+H170K, Q90K+Q120K+T133K, Q90K+Q120K+S159K, Q90K+Q120K+H170K, Q90K+T133K+S159K, Q90K+T133K+H170K, Q90+S159K+H170K, T105K+Q120K+T133K, T105K+Q120K+S159K, T105K+Q120K+H170K, T105K+T133K+S159K, T105K+T133K+H170K, T105K+S159K+H170K, Q120K+T133K+S159K, Q120K+T133K+H170K, Q120K+S159K+H170K, T133K+S159K+H170K, Q70K+Q90K+T105K+Q120K, Q70K+Q90K+T105K+T133K, Q70K+Q90K+T105K+S159K, Q70K+Q90K+T105K+H170K, Q70K+Q90K+Q120K+T133K, Q70K+Q90K+Q120K+S159K, Q70K+Q90K+Q120K+H170K, Q70K+Q90K+T133K+S159K, Q70K+Q90K+T133K+H170K, Q70K+Q90K+S159K+H170K, Q70K+T105K+Q120K+T133K, Q70K+T105K+Q120K+S159K, Q70K+T105K+Q120K+H170K, Q70K+T105K+T133K+S159K, Q70K+T105K+T133K+H170K, Q70K+T105K+S159K+H170K, Q70K+Q120K+T133K+S159K, Q70K+Q120K+T133K+H170K, Q70K+T133K+S159K+H170K, Q90K+T105K+Q120K+T133K, Q90K+T105K+Q120K+S159K, Q90K+T105K+Q120K+H170K, Q90K+T105+T133K+S159K, Q90K+T105+T133K+H170K, Q90K+T105+S159K+H170K, Q90K+Q120K+T133K+S159K, Q90K+Q120K+T133K+H170K, Q90K+Q120K+S159K+H170K, Q90K+T133K+S159K+H170K, T105K+Q120K+T133K+S159K, T105K+Q120K+T133K+H170K, T105K+Q120K+S159K+H170K, T105K+T133K+S159K+H170K or Q120K+T133K+S159K+H170K. In any of the variants listed above, the substitution H170K may instead be H170Q or H170R. Particularly preferred substitutions to introduce a lysine include one or both of T105K and S159K.

In a further embodiment, the G-CSF polypeptide may be altered to produce a G-CSF variant in which one or more of the native lysine residues in positions 16, 23, 34 and 40 is removed in order to avoid PEGylation at these positions. For example, one or more of these lysine residues may be removed by way of substitution, preferably with an arginine or glutamine residue, more preferably with an arginine residue. Preferably, one or more of the lysine residues at positions 16, 34 and 40 are removed by way of substitution, more preferably two or three of these lysine are removed, and most preferably all three of the lysines at this position are removed by substitution. Thus, in a preferred embodiment the G-CSF variant comprises the sequence of SEQ ID NO: 1 with at least one substitution selected from the group consisting of K16R, K16Q, K34R, K34Q, K40R and K40Q. In a particularly preferred embodiment, the variant comprises the substitutions K16R+K34R+K40R.

In a more preferred embodiment, the G-CSF variant comprises at least one substitution to introduce a lysine residue together with at least one substitution to remove a lysine residue as explained above.

In another embodiment, the multi-PEGylated G-CSF variant comprises a substitution of one or more of the lysine residues at positions 16, 34, and 40, such as with an arginine or a glutamine residue, e.g., an arginine residue, and one or more substitution selected from Q70K, Q90K, T105K, Q120K, T133K, and S159K, and is conjugated to 2-6, such as 2-4, polyethylene glycol moieties each with a molecular weight of about 1000-10,000 Da.

In another embodiment, the multi-PEGylated G-CSF variant comprises one or more substitution selected from K16R, K34R, and K40R, and one or more substitution selected from Q70K, Q90K, T105K, Q120K, T133K, and S159K, and is conjugated to 2-6, such as 2-4, polyethylene glycol moieties each with a molecular weight of about 1000-10,000 Da.

In another embodiment, the multi-PEGylated G-CSF variant comprises a substitution of one or more of the lysine residues at positions 16, 34, and 40, such as with an arginine or a glutamine residue, e.g., an arginine residue, and at least one substitution selected from T105K and S159K, and is conjugated to 2-6, such as 2-4, polyethylene glycol moieties each with a molecular weight of about 1000-10,000 Da.

In another embodiment, the multi-PEGylated G-CSF variant comprises one or more substitution selected from K16R, K34R, and K40R, and at least one substitution selected from T105K and S159K, and is conjugated to 2-6, such as 2-4, polyethylene glycol moieties each with a molecular weight of about 1000-10,000 Da.

In a particular embodiment the multi-PEGylated G-CSF variant comprises the substitutions K16R, K34R, K40R, T105K and S159K and is conjugated to 2-6, such as 2-4, polyethylene glycol moieties with a molecular weight of about 1000-10,000 Da.

In particular, the multi-PEGylated G-CSF variant may have 2-6, typically 2-5, such as 2-4, polyethylene glycol moieties with a molecular weight of about 5000-6000 Da attached, e.g. mPEG with a molecular weight of about 5 kDa. Preferably, the multi-PEGylated G-CSF variant has 2-4 polyethylene glycol moieties with a molecular weight of about 5000-6000 Da attached, e.g. 5 kDa mPEG. A particularly preferred multi-PEGylated G-CSF variant that is suitable for use in the method of the invention comprises the substitutions K16R, K34R, K40R, T105K and S159K and contains 2-4 PEG moieties each with a molecular weight of about 5 kDa, primarily 3 such PEG moieties.

In another embodiment, the multi-PEGylated G-CSF variant may be produced so as to have only a single number of PEG moieties attached, e.g. either 2, 3, 4 or 5 PEG moieties per conjugate, or to have a desired mix of conjugates with different numbers of PEG moieties attached, e.g. a mix of conjugates having 2-5, 2-4, 3-5, 3-4, 4-6, 4-5 or 5-6 attached PEG moieties. As indicated above, an example of a preferred conjugate mix is one having 2-4 PEG moieties of about 5 kDa, for example a conjugate having primarily 3 PEG moieties attached per conjugate but with a small proportion of the conjugates having either 2 or 4 PEG moieties attached.

It will be understood that a conjugate having a specific number of attached PEG moieties, or a mix of conjugates having a defined range of numbers of attached PEG moieties, may be obtained by choosing suitable PEGylation conditions and optionally by using subsequent purification to separate conjugates having the desired number of PEG moieties. Examples of methods for separation of G-CSF conjugates with different numbers of PEG moieties attached as well as methods for determining the number of PEG moieties attached are described, e.g. in WO 01/51510 and WO 03/006501, both of which are incorporated herein by reference. For purposes of the present invention, a conjugate may be considered to have a given number of attached PEG moieties if separation on an SDS-PAGE gel shows no or only insignificant bands other than the band(s) corresponding to the given number(s) of PEG moieties. For example, a sample of a conjugate is considered to have 3 attached PEG groups if an SDS-PAGE gel on which the sample has been run shows a major bands corresponding to 3 PEG groups, respectively, and only insignificant bands or, preferably, no bands corresponding to 2 or 4 PEG groups.

In some cases, amine-specific activated PEG derivatives such as mPEG-SPA may not attach exclusively to the N-terminus and the ε-amino groups of lysine residues via an amide bond, but may also attach to the hydroxy group of a serine, tyrosine or threonine residue via an ester bond. As a result, the PEGylated proteins may not have a sufficient degree of uniformity and may contain a number of different PEG isomers other than those that were intended. Such PEG moieties bound via an ester bond will typically be labile and can be removed by the method described in U.S. Provisional Patent Application No. 60/686,726, incorporated herein by reference, which involves subjecting the PEGylated polypeptide to an elevated pH for a period of time sufficient to remove the labile PEG moieties attached to a hydroxy group. This method is also described in U.S. Ser. No. 11/420,546 (U.S. Pat. No. 7,381,805) and WO 2006/128460, both of which are incorporated herein by reference.

In a preferred embodiment, the multi-PEGylated G-CSF variant is a mixture of positional PEG isomer species. As used herein, the term “positional PEG isomer” of a protein refers to different PEGylated forms of the protein where PEG groups are located at different amino acid positions of the protein. A preferred multi-PEGylated G-CSF variant employed in the practice of the present invention is a mixture of lysine/N-terminal PEG isomers. The term “lysine/N-terminal PEG isomer” of a protein means that the PEG groups are attached to the amino-terminal of the protein and/or to epsilon amino groups of lysine residues in the protein. For example, the phrase “lysine/N-terminal positional PEG isomers having 3 attached PEG moieties”, as applied to G-CSF, means a mixture of G-CSF positional PEG isomers in which three PEG groups are attached to epsilon amino groups of lysine residues and/or to the N-terminus of the protein. Typically, a “lysine/N-terminal positional PEG isomer having 3 attached PEG moieties” will have two PEG moieties attached to lysine residues and one PEG moiety attached to the N-terminus. Analysis of the positional PEG isomers may be performed using cation exchange HPLC as described in WO 2006/128460, which is incorporated herein by reference.

Typically, the mixture of positional PEG isomer species is a substantially purified mixture of lysine/N-terminal positional PEG isomers. A “substantially purified mixture of lysine/N-terminal positional PEG isomers” of a polypeptide refers to a mixture of lysine/N-terminal positional PEG isomers which has been subjected to a chromatographic or other purification procedure in order to remove impurities such as non-lysine/N-terminal positional PEG isomers. The “substantially purified mixture of lysine/N-terminal positional PEG isomers” will, for example, be free of most labile PEG moieties attached to a hydroxyl group that would otherwise be present in the absence of a partial de-PEGylation step and subsequent purification as described herein, and will typically contain less than about 20% polypeptides containing a labile PEG moiety attached to a hydroxyl group, more typically less than about 15%. Preferably, there will be less than about 10% polypeptides containing a labile PEG moiety attached to a hydroxyl group, for example, less than about 5%.

Preferably, the mixture of positional PEG isomer species is a homogeneous mixture of positional PEG isomers of a G-CSF variant. The term “homogeneous mixture of positional PEG isomers of a polypeptide (G-CSF) variant” means that the polypeptide moiety of the different positional PEG isomers is the same. This means that the different positional PEG isomers of the mixture are all based on a single polypeptide variant sequence. For example, a homogeneous mixture of positional PEG isomers of a PEGylated G-CSF polypeptide variant means that different positional PEG isomers of the mixture are based on a single G-CSF polypeptide variant.

Typically, the homogeneous mixture of positional PEG isomers of a G-CSF variant exhibits substantial uniformity. As used herein, “uniformity” refers to the homogeneity of a PEGylated polypeptide in terms of the number of different positional isomers, i.e., different polypeptide isomers containing different numbers of PEG moieties attached at different positions, as well as the relative distribution of these positional isomers. For pharmaceutical polypeptides intended for therapeutic use in humans or animals, it is generally desirable that the number of different positional PEG isomers and different PEGylated species is minimized.

The term, “partial de-PEGylation” refers herein to the removal of labile PEG moieties attached to a hydroxyl group, while PEG moieties that are more stably attached to the N-terminal or the amino group of a lysine residue remain intact. The method for carrying out this process is described in U.S. Ser. No. 60/686,726, U.S. Ser. No. 11/420,546 (U.S. Pat. No. 7,381,805), and WO 2006/128460, all of which are incorporated herein by reference.

In a preferred embodiment (referred to as “Maxy-G” in the examples hereinbelow), the multi-PEGylated G-CSF variant is a mixture of positional PEG isomers where the G-CSF variant component has the amino acid sequence of SEQ ID NO: 1 with the substitutions K16R, K34R, K40R, T105K and S159K (relative to SEQ ID NO: 1), and where at least 80% of the mixture has at least 2 species of positional PEG isomers each having 3 attached PEG moieties, where one of the isomers has PEG groups attached at the N-terminal, Lys 23 and Lys 159 and the other isomer has PEG groups attached at the N-terminal, Lys105 and Lys159. The multi-PEGylated G-CSF variant referred to as “Maxy-G” herein comprises PEG moieties that are mPEG-SPA (Nektar), each having an average molecular weight of 5000 Da.

For all the embodiments described above, the G-CSF variant and the multi-PEGylated G-CSF variant may optionally include a methionine residue added to the N-terminus.

In further embodiments, the multi-PEGylated G-CSF variant to be administered according to the invention may be prepared as described in any of the following, all of which are incorporated herein by reference:
WO 89/05824 (lysine-depleted variants of G-CSF)
U.S. Pat. No. 5,824,778 (G-CSF having at least one PEG molecule covalently attached to at least one amino acid of the polypeptide through a carboxyl group of said amino acid)
WO 99/03887 (PEGylated cysteine variants of G-CSF)
WO 2005/055946 (“glyco-PEGylated” G-CSF conjugates with PEG moieties linked via an intact glycosyl linking group)
WO 2005/070138 (G-CSF polypeptides comprising a mutant peptide sequence encoding an O-linked glycosylation site that does not exist in the corresponding wild-type polypeptide).
US 2005/0114037 A1 (G-CSF with at least one polymeric moiety attached at least one of a number of different specified amino acid positions)

In another embodiment, the multi-PEGylated G-CSF variant to be administered according to the invention exhibits an improved pharmacokinetic property, such as an increased serum half-life and/or an increased AUC, compared to the mono-PEGylated G-CSF Neulasta®. Preferably, the multi-PEGylated G-CSF variant exhibits a serum half-life or an AUC increased by at least about 1.2× of the serum half-life or AUC of Neulasta®, e.g. increased by at least about 1.4×, such as by at least about 1.5×, e.g. by at least about 1.6×, such as by at least about 1.8×, e.g. by at least about 2.0×, 2.5×, 3×, 5×, or 10× that of G-CSF Neulasta®.

### Classes of Chemotherapeutic Agents

Chemotherapeutic agents are generally categorized according to their mechanism of action, chemical type and/or biological source. Provided below is a description of various classes of chemotherapeutic agents and agents used in cancer chemotherapy which are examples of agents and treatment protocols suitable for use in the methods of the invention.

Alkylating Agents

Alkylating agents kill cancer cells by reacting with cellular DNA, resulting in cross-linking or strand breaks which inhibit base pairing, replication, and/or transcription of tumor cell genes. Alkylating agents are active in every stage of the cell cycle and are most active in the resting phase. There are several types of alkylating agents used in chemotherapy, including, but not limited to:
Mustard gas derivatives, such as Cyclophosphamide, Chlorambucil, Ifosfamide, Mechlorethamine, and Melphalan.
Ethylenimines, such as Hexamethylmelamine and Thiotepa.
Alkylsulfonates such as Busulfan.
Hydrazines and Triazines such as Altretamine, Dacarbazine, Procarbazine, and Temozolomide.
Nitrosureas such as Carmustine, Lomustine and Streptozocin.
Inorganic metal complex agents (e.g., metal complexes of platinum, palladium or ruthenium), such as Cisplatin, Carboplatin, and Oxaliplatin.

The alkylating agents are very powerful chemotherapeutics and are used to treat most every type of cancer, solid tumors as well as hematologic malignancies. Unlike most types of chemotherapeutic agents, nitrosureas can cross the blood-brain barrier, and therefore may be particularly useful in treating brain tumors.

Plant Alkaloids

The plant alkaloids are a class of chemotherapeutic agents isolated from various plants. Taxanes (derived from the bark of certain yew trees) and the vinca alkaloids (derived from periwinkle plants) are antimicrotubule agents. Camptothecan analogs (derived from the Camptotheca acuminata tree) and podophyllotoxins (derived from mandrake plants) are topoisomerase inhibitors. The plant alkaloids are cell-cycle specific and attack the cells during various phases of cell division. Plant alkaloids used in chemotherapy include, but are not limited to:
Antimicrotubule agents, such as taxanes (e.g., Docetaxel and Paclitaxel) and vinca alkaloids (e.g., Vinblastine, Vincristine, and Vinorelbine).
Topoisomerase inhibitors, such as camptothecan analogs (e.g., Irinotecan and Topotecan) and podophyllotoxins (e.g., Etoposide and Tenisopide).

Antitumor Antibiotics

Antitumor antibiotics are a class of chemotherapeutic agents produced by various species of Streptomyces. Mechanisms of action of antitumor antibiotics include inhibition of topoisomerases and/or generation of free oxygen radicals which result in DNA strand breaks and inhibition of DNA synthesis. Antitumor antibiotics used in chemotherapy include, but are not limited to:
Anthracyclines, such as Daunorubicin, Doxorubicin, Epirubicin, Idarubicin, and Mitoxantrone.
Chromomycins, such as Dactinomycin and Plicamycin.
Other antitumor antibiotics such as Bleomycin and Mitomycin.

Antimetabolites

Antimetabolites are inhibitors (antagonists) of molecules involved in cellular metabolism. Antimetabolites are generally cell-cycle specific, and are classified according to the substances with which they interfere. Antimetabolites used in chemotherapy include, but are not limited to:
Folic acid antagonists, such as Methotrexate.
Pyrimidine antagonists, such as Capecitabine, Cytarabine, 5-Fluorouracil (5-FU), Foxuridine, and Gemcitabine.
Purine antagonists, such as 6-Mercaptopurine and 6-Thioguanine.
Adenosine deaminase inhibitors, such as Cladribine, Fludarabine, Nelarabine and Pentostatin.
Ribonucleotide reductase inhibitors, such as Hydroxyurea.

Topoisomerase Inhibitors

Topoisomerase inhibitors are a class of molecules which interfere with the action of the topoisomerase enzymes topoisomerase I and II and inhibit DNA replication. Topoisomerase inhibitors used in chemotherapy include, but are not limited to:
Topoisomerase I inhibitors, such as Ironotecan and Topotecan
Topoisomerase II inhibitors, such as Amsacrine, Etoposide, Etoposide Phosphate, and Teniposide

Miscellaneous Chemotherapeutic Agents

Additional types of compounds used in chemotherapy include, but are not limited to:
Andrenolytic agents, such as the adrenocortical steroid inhibitor Mitotane
Enzymes, such as Asparaginase and Pegaspargase.
Retinoids, such as Bexarotene, Isotretinoin, and Tretinoin (All-Trans-Retinoic Acid).

### Chemotherapeutic Regimens in Clinical Practice

In some cancers, chemotherapies employing single agents (single-agent regimens) are effective, but in some instances better outcomes are achieved by treatments involving combination chemotherapy, which involves simultaneous or sequential administration of two or more agents, often from different chemotherapeutic classes or sub-classes such as those described above. Combination chemotherapy has several advantages over single-agent treatment: it provides for maximal cell kill within the range of toxicities tolerated by the host for each individual drug, it allows for a broader range of interactions between drugs and tumor cells in a heterogeneous tumor population, and it may prevent or slow the development of drug resistance. Combination chemotherapy employing single agents administered in an accelerated defined sequence (“dose-dense therapy”) is also employed for treatment of certain types of cancers.

Numerous single-agent and combination chemotherapeutic regimens have been employed for treatment of specific solid tumors and hematologic malignancies. The following are non-limiting examples of various chemotherapeutic regimens typically used for different types of cancer which may be employed in the methods of the invention. Detailed guidance concerning dosages, timing and duration of treatment may be found, for example, in clinical oncology reference books known to those of skill in the art, such as Chu, E. and DeVita, V. T. Physician's Cancer Chemotherapy Drug Manual 2005, Jones and Bartlett Publishers, Sudbury, Mass. (2005); and Abraham, J. et al. (eds.) Bethesda Handbook of Clinical Oncology, 2^{nd} Edition, Lippincott Williams & Wilkins, Philadelphia, Pa. (2005).

### Breast Cancer

Doxorubicin, Cyclophosphamide; followed by Docetaxel or Paclitaxel (AC+T regimen)
Doxorubicin, Paclitaxel (AT regimen)
Docetaxel, Doxorubicin, Cyclophosphamide (TAC regimen)
Doxorubicin, Cyclophosphamide (AC regimen)
Docetaxel
Paclitaxel
Docetaxel, Capecitabine (DX regimen)
Cyclophosphamide, Epirubicin, Fluorouracil
Cyclophosphamide, Methotrexate, Fluorouracil (CMF regimen)
Cyclophosphamide, Doxorubicin, Fluorouracil (CAF regimen)

### Lung Cancer (Non-Small Cell)

Gemcitabine, Ifosfamide, Vinorelbine (VIG regimen)
Docetaxel, Carboplatin (DP regimen)
Cisplatin, Paclitaxel (TC regimen)
Carboplatin, Paclitaxel
Carboplatin, Etoposide

### Lung Cancer (Small Cell)

Cyclophosphamide, Doxorubicin, Etoposide (CAE regimen)
Topotecan
Topotecan, Paclitaxel (TopT regimen)
Cisplatin, Topotecan (TopC regimen)
Etoposide, Carboplatin (EP regimen)

### Colorectal Cancer

Fluorouracil, Oxaliplatin
Fluorouracil, Irinotecan

### Uterine/Ovarian Cancers

Topotecan
Paclitaxel
Docetaxel
Carboplatin, Paclitaxel

### Non-Hodgkin's Lymphoma (NHL)

Vincristine, Doxorubicin, Prednisolone, Etoposide, Cyclophosphamide, Bleomycin (VAPEC-B regimen)
Doxorubicin or Mitoxantrone, Cyclophosphamide, Vindesine, Bleomycin (A(N)CVB regimen)
Dexamethasone, Cisplatin, Cytarabine (DHAP regimen)
Etoposide, Methylprednisolone, Cisplatin, Cytarabine (ESHAP regimen)
Doxorubicin, Cyclophosphamide, Vincristine, Prednisone (ACOD regimen)
Fludarabine, Mitoxantrone (FM regimen)
Cyclophosphamide, Doxorubicin, Vincristine, Prednisone, (CHOP regimen)
Cyclophosphamide, Doxorubicin, Vincristine, Prednisone, Rituximab, (CHOP-R regimen)

### Hodgkin's Disease

Mechlorethamine, Doxorubicin, Vinblastine, Bleomycin, Etoposide, Prednisone (Stanford V)
Bleomycin Dacarbazine, Doxorubicin, Vinblastine

Patients undergoing cancer chemotherapy generally exhibit moderate to severe neutropenia after treatment. The extent and duration of neutropenia is significantly diminished by same-day administration of a multi-PEGylated G-CSF variant in accordance with the methods of the present invention.

### Dosages

The dosage of the multi-PEGylated G-CSF variant administered according to the invention will generally be approximately the same order of magnitude as the current recommended dosage for PEG-filgrastim (Neulasta®), which is 6 mg per adult patient. An appropriate dose of the multi-PEGylated G-CSF variant is therefore contemplated to be in the range of from about 1 mg to about 15 mg, such as from about 2 mg to about 15 mg, e.g. from about 3 mg to about 12 mg. A suitable dose may thus be, for example, about 3 mg, about 6 mg, or about 9 mg. As illustrated in Example 2, the decrease in duration of leukopenia, duration of neutropenia, and ANC time to recovery (TTR) occurred at lower doses as compared to the similar effect for mono-PEGylated hG-CSF (Neulasta® PEG-Filgrastim). Accordingly, it is contemplated that the multi-PEGylated G-CSF variant may be administered on the same day as chemotherapy in a dosage that is less than 6 mg per adult patient, typically in a dose of from about 1 mg to about 5 mg, or from about 2 mg to about 4 mg, or from about 3 mg to about 4 mg. The lower dose may be 1 mg, 2 mg, 3 mg, 4 mg, or 5 g per adult patient. In each case, the dosages are expressed as a standard dose per patient, where the patient is an adult or otherwise weighs at least 45 kg. Alternatively, dosage may be determined according to the weight of the patient, such that an appropriate dose of the multi-PEGylated G-CSF variant is contemplated to be in the range of from about 5 or 10 μg/kg to about 200 μg/kg, such as about 25 μg/kg to about 200 μg/kg, such as about 50 μg/kg to about 150 μg/kg, e.g. from about 75 μg/kg to about 125 μg/kg. A suitable dose may thus be, for example, about 25 μg/kg, about 50 μg/kg, about 75 μg/kg, about 100 μg/kg, about 125 μg/kg or about 150 μg/kg. In carrying out the practice of the present invention, suitable doses include lower doses in the range of from about 5 or 10 μg/kg to less than 100 μg/kg, from about either 5 or 10 μg/kg to less than about either 60, 70, 80, or 90 μg/kg. Further suitable lower doses may be in the range of from about 5 or 10 μg/kg to about 50 μg/kg, or about 5 or 10 μg/kg to about 40 μg/kg, or about 5 or 10 μg/kg to about 30 μg/kg.

### Pharmaceutical Compositions

The multi-PEGylated G-CSF variant administered according to the present invention is administered in a composition including one or more pharmaceutically acceptable carriers or excipients. The multi-PEGylated G-CSF variant can be formulated into pharmaceutical compositions in a manner known per se in the art to result in a pharmaceutical that is sufficiently storage-stable and is suitable for administration to humans or animals. The pharmaceutical composition may be formulated in a variety of forms, including as a liquid or gel, or lyophilized, or any other suitable form. The preferred form will depend upon the particular indication being treated and will be apparent to one of skill in the art.

“Pharmaceutically acceptable” means a carrier or excipient that at the dosages and concentrations employed does not cause any untoward effects in the patients to whom it is administered. Such pharmaceutically acceptable carriers and excipients are well known in the art (see, e.g., Remington's Pharmaceutical Sciences, 18th edition, A. R. Gennaro, Ed., Mack Publishing Company (1990); Pharmaceutical Formulation Development of Peptides and Proteins, S. Frokjaer and L. Hovgaard, Eds., Taylor & Francis (2000); and Handbook of Pharmaceutical Excipients, 3rd edition, A. Kibbe, Ed., Pharmaceutical Press (2000)).

Parenteral Compositions

An example of a pharmaceutical composition is a solution designed for parenteral administration, e.g. by the subcutaneous route. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives are added to retard microbial growth, and are typically added in amounts of about 0.2%-1% (w/v). Suitable preservatives for use with the present invention include phenol, benzyl alcohol, meta-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g. starch), chelating agents (e.g. EDTA), antioxidants (e.g., ascorbic acid, methionine, vitamin E) and cosolvents.

The active ingredient may also be entrapped in microcapsules prepared, for example, by coacervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

Parenteral formulations to be used for in vivo administration must be sterile. This is readily accomplished, for example, by filtration through sterile filtration membranes.

The invention is further described by the following non-limiting examples.

### EXAMPLES

### Example 1

### Measurement of Pharmacokinetics of PEGylated G-CSF Molecules in Normopenic Rats

The pharmacokinetics of a PEGylated G-CSF molecule in normopenic (i.e., healthy) rats is measured as follows. Male Sprague Dawley rats (7 weeks old) are used. On the day of administration, the weights of the animals are measured (generally, 280 to 310 gram per animal). 100 μg per kg body weight of the PEGylated G-CSF samples are each injected intravenously into the tail vein of three rats. At 1 minute, 30 minutes, 1, 2, 4, 6, and 24 hours after the injection, 500 μl of blood is withdrawn from each rat while under CO₂-anesthesia. The blood samples are stored at room temperature for 11 hours followed by isolation of serum by centrifugation (4° C., 18000×g for 5 minutes). The serum samples are stored at −80° C. until the day of analysis. After thawing the samples on ice, the serum concentration of G-CSF is quantified either by a G-CSF in vitro activity assay (such as the in vitro assay for G-CSF activity using the NFS-60 cell line as described in Hammerling et al. (1995) J. Pharm. Biomed. Anal. 13(1):9-20), which is incorporated herein by reference, or by ELISA, for example using a using a commercial ELISA kit (such as human G-CSF DuoSet ELISA; R&D Systems, Minneapolis, Minn.).

The following pharmacokinetic parameters are determined:

kₑₗ: The apparent terminal elimination rate constant, calculated from a semi-log plot of the serum concentration versus time curve. The parameter is calculated by linear least-squares regression analysis using the maximum number of points in the terminal log-linear phase (e.g. three or more non-zero serum concentration values).

t_{1/2}: Apparent terminal elimination half-life (also termed “serum half-life”) calculated as: ln(2)/kₑₗ.

AUC₀₋ₜ: The area under the serum concentration versus time curve, from time 0 to the last measurable concentration, as calculated by the linear trapezoidal method.

AUC_{inf}: The area under the serum concentration versus time curve, from time 0 to infinity, calculated as the sum of the AUC₍₀₋ₜ₎ plus the ratio of the last measurable serum concentration to the elimination rate constant.

### Example 2

### Same-Day Administration of PEGylated G-CSF and Cyclophosphamide

A study was performed to evaluate the ability of same-day administration of a multi-PEGylated G-CSF variant to counteract or minimize the period of neutropenia induced by Cyclophosphamide, a chemotherapeutic agent, in male rats, and to compare the effect to that of the mono-PEGylated G-CSF, Neulasta® (pegfilgrastim).

### Materials and Methods

Animals: Fifty-four (54) male, Sprague Dawley rats (M&B Taconic) weighing approximately 190 g were used in the study.

Chemotherapeutic agent: Cyclophosphamide (CPA) was prepared at a concentration of 33.33 mg/mL by dissolving 1000 mg Sendoxan (Baxter Oncology, Halle, Germany) in 30 ml 0.9% normal saline.

Reference mono-PEGylated G-CSF: Neulasta® (pegfilgrastim; Amgen, Thousand Oaks, Calif., USA) was formulated in 10 mM Na-acetate containing sorbitol (50 mg/mL) and Tween-20 (33 μg/mL) at concentrations of 100, 300 and 1000 μg/mL.

Test multi-PEGylated G-CSF variant: The exemplary multi-PEGylated G-CSF variant administered according to the invention (“Maxy-G”) was a variant of human G-CSF (SEQ ID NO:1) with the substitutions K16R, K34R, K40R, T105K and S159K. The G-CSF variant was produced in CHO-K1 cells and PEGylated using mPEG-SPA 5000 (Nektar Therapeutics) as described in WO 03/006501, which is incorporated herein by reference, to result in a multi-PEGylated G-CSF variant having 4-5 PEG moieties per G-CSF molecule. In order to provide a more stable and uniform PEGylation, labile PEG moieties were removed by subjecting the multi-PEGylated G-CSF variant to partial de-PEGylation at an elevated pH as described in U.S. Provisional Patent Application No. 60/686,726 (incorporated herein by reference). The partial de-PEGylation method is also described in related patent publications U.S. Ser. No. 11/420,546 (U.S. Pat. No. 7,381,805) and WO 2006/128460, both of which are incorporated herein by reference. The result was a uniform and stable multi-PEGylated G-CSF variant having primarily three attached 5 kDa PEG moieties per G-CSF molecule. Maxy-G was prepared in a vehicle solution of 10 mM Na-Acetate containing 43 mg/mL mannitol (pH=4.0) at concentrations of 30, 100, or 300 mg/mL Maxy-G.

Vehicle: Aqueous solution of 10 mM Na-Acetate containing 43 mg/mL mannitol (pH 4.0).

Experimental Procedures: On day 1, the animals were assigned to one of seven groups (vehicle; 30, 100 or 300 μg/kg Maxy-G; or 100, 300 or 1000 μg/kg Neulasta®) and a tail vein blood sample was collected from each animal for use as a baseline (t0) determination. On day 2, the animals were individually weighed and were administered CPA intraperitoneally (i.p.) at a dose of 90 mg/kg to induce neutropenia. Two hours later, Maxy-G, Neulasta® or vehicle was administered subcutaneously in the scruff of the neck at an injection volume of approximately 700 μl (2.7 mL/kg body weight). On days 3-13, tail vein blood samples were obtained from each animal for pharmacokinetic determination. The final pharmacokinetic sample was obtained on Day 16. Immediately after the last blood sample collection, all animals were humanely euthanized

Each animal was weighed three more times during the course of the study, on days 3, 7 and 14.

During the study, clinical observations were performed twice daily (except on weekends when clinical observations were performed once daily). During the first 10 hours following drug or vehicle treatment, clinical observations were performed four times. Food and water consumption was monitored to assure that the animals were eating and drinking, although these data were not collected.

Blood Collection: Blood samples were taken for pharmacodynamic measurements at 24-hour intervals on days 3-14 (24, 48, 72, 96, 120, 144, 168, 192, 216, 240 and 264 hours following treatment, respectively) and a final sample was collected on day 16.

Pharmacodynamic Determinations: The effect of Maxy-G and Neulasta® on white blood cell count (WBC) and other hematological parameters was determined. The relative neutrophil count was manually performed using blood smears. The absolute neutrophil count (ANC) was manually calculated from the relative level of ANC vs. WBC.

Definitions of leukopenia, neutropenia, duration of leukopenia, duration of neutropenia and the time to recovery: Leukopenia is defined as a WBC count below 4×10⁹ cells/L (Merck Manual 2006). When the absolute neutrophil count (ANC) is measured, neutropenia is defined as an ANC count below 0.5×10⁹ cells/L (Merck Manual 2006).

The days of leukopenia is defined as the number of days when the individual WBC count is below 4×10⁹ cells/L after CPA, calculated on the basis of the samples taken every 24 hours.

The days of severe neutropenia is defined as the number of days when the individual ANC count is below 0.5×10⁹ cells/L after CPA, calculated on the basis of the samples taken every 24 hours.

The time to recovery is defined as the number of days starting from the CPA administration until the first of 2 consecutive days for individual animals with counts at or above 4×10⁹ WBC cells/L or 0.5×10⁹ ANC cells/L, respectively.

### Results

Treatment with CPA results in a profound and prolonged leukopenia and neutropenia in rats, which is characterized by decreased circulating levels of WBC (<4.0×10⁹ cells/L) and ANC (<0.5×10⁹ cells/L) respectively. In the present study, this effect is manifested in the vehicle-treated group three or four days after treatment with CPA and the leukopenia is maintained for 5.5±1.6 days while the neutropenia is maintained for 6.3±1.2 days (Table 1). The administration of Maxy-G or Neulasta® two-hours after treatment with CPA does not block the development of the CPA-induced leukopenia or neutropenia. However, both drugs significantly attenuate the duration of the response (Table 1), leading to significantly faster time-to-recovery (“TTR”; Table 2).

The ability of Maxy-G and Neulasta® to affect a recovery from the CPA-induced leukopenic and neutropenic responses appeared to be dose-dependent. As shown in Table 1, the number of days in which Maxy-G-treated rats are leukopenic following treatment with CPA demonstrates dose-dependency, with significantly fewer days of leukopenia (p<0.05) after the administration of 100 or 300 μg/kg. Similarly, the effect of Maxy-G on CPA-induced neutropenia demonstrates dose dependency with significantly fewer days of neutropenia (to 51% of vehicle, p<0.05), noted following the administration with as little as 30 μg/kg Maxy-G. While treatment with Neulasta® also displays a dose-dependent effect against both CPA-induced leukopenia and neutropenia, about 3-fold higher doses are required to achieve similar effects as with Maxy-G (Table 1).

TABLE 1
The effect of treatment with Maxy-G and Neulasta ® on
days of leukopenia and neutropenia following the intraperitoneal
administration of CPA to normopenic rats.

| | | | Days of severe | |
|---|---|---|---|---|
| | Days of leukopenia | | neutropenia | |
| | (Days with | | (Days with | |
| Compound | WBC < 4.0 × 10⁹/L) | N | ANC < 0.5 × 10⁹/L) | N |
| Vehicle | 5.5 ± 1.6 | 6 | 6.3 ± 1.2 | 6 |
| Maxy-G | 5.2 ± 0.8 | 6 | 3.2 ± 1.0* | 6 |
| (30 μg/kg) | | | | |
| Maxy-G | 2.7 ± 0.5*^{abc} | 6 | 1.8 ± 0.8*^{a} | 6 |
| (100 μg/kg) | | | | |
| Maxy-G | 2.3 ± 1.0*^{abc} | 6 | 1.3 ± 1.2*^{c} | 6 |
| (300 μg/kg) | | | | |
| Neulasta ® | 4.8 ± 0.8 | 6 | 3.8 ± 1.0* | 6 |
| (100 μg/kg) | | | | |
| Neulasta ® | 4.2 ± 1.0 | 6 | 2.0 ± 0.6*^{a} | 6 |
| (300 μg/kg) | | | | |
| Neulasta ® | 2.2 ± 1.0*^{abc} | 6 | 1.7 ± 0.5*^{ac} | 6 |
| (1000 μg/kg) | | | | |

Mean ± SD; Statistical difference (P) from vehicle is:
*P < 0.05, from Neulasta ® (100 μg/kg):
^{a}P > 0.05, from Neulasta ® (300 μg/kg):
^{b}P > 0.05, and from Maxy-G (30 μg/kg):
^{c}P < 0.05.

A dose-response relationship following treatment with Maxy-G or Neulasta® is also seen when TTR is evaluated. As shown in Table 2, vehicle-treated animals are normopenic (non-neutropenic) with respect to circulating leukocyte and neutrophil levels approximately 8.3 to 9.2 days after CPA treatment, respectively. Treatment with Maxy-G or Neulasta® two hours after the administration of CPA results in a return to normal levels of both white blood cell subtypes (WBC and ANC) significantly more rapidly than occurs in the vehicle treated group. As is true with the duration of the leukopenic and neutropenic responses, the TTR response to Maxy-G occurs at about 3-fold lower doses than is required for a similar effect following treatment with Neulasta®.

TABLE 2
The effect of treatment with Maxy-G or Neulasta ® on
the time-to-recovery from leukopenia and neutropenia in rats
administered CPA.

| | WBC | | ANC | |
|---|---|---|---|---|
| | Time to recovery | | Time to recovery | |
| Compound | (Days) | N | (Days) | N |
| Vehicle | 8.3 ± 0.5 | 6 | 9.2 ± 0.4 | 6 |
| Maxy-G | 8.0 ± 0.6 | 6 | 6.7 ± 0.5* | 6 |
| (30 μg/kg) | | | | |
| Maxy-G | 6.0 ± 0* | 6 | 5.2 ± 0.4*^{a} | 6 |
| (100 μg/kg) | | | | |
| Maxy-G | 5.7 ± 0.5** | 6 | 4.5 ± 1.8*^{ac} | 6 |
| (300 μg/kg) | | | | |
| Neulasta ® | 8.3 ± 0.5 | 6 | 7.5 ± 0.8* | 6 |
| (100 μg/kg) | | | | |
| Neulasta ® | 7.5 ± 0.8 | 6 | 5.0 ± 0.6*^{ac} | 6 |
| (300 μg/kg) | | | | |
| Neulasta ® | 5.8 ± 1.2** | 6 | 5.0 ± 0.0*^{ac} | 6 |
| (1000 μg/kg) | | | | |

Mean ± SD; Statistical difference (P) from vehicle is:
*P < 0.05, from Neulasta ® (100 μg/kg):
^{a}P < 0.05, from Neulasta ® (300 μg/kg):
^{b}P < 0.05, and from Maxy-G (30 μg/kg):
^{c}P < 0.05

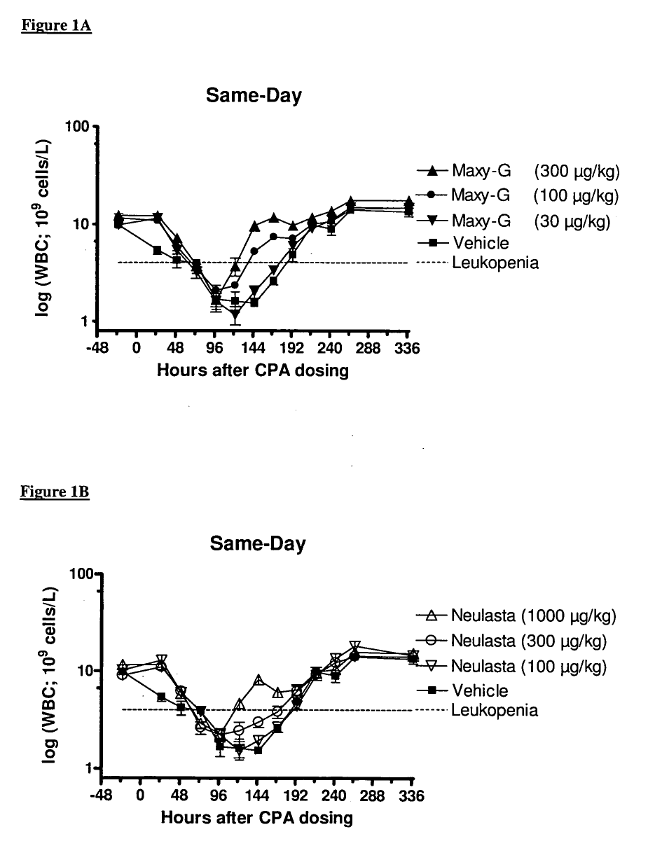

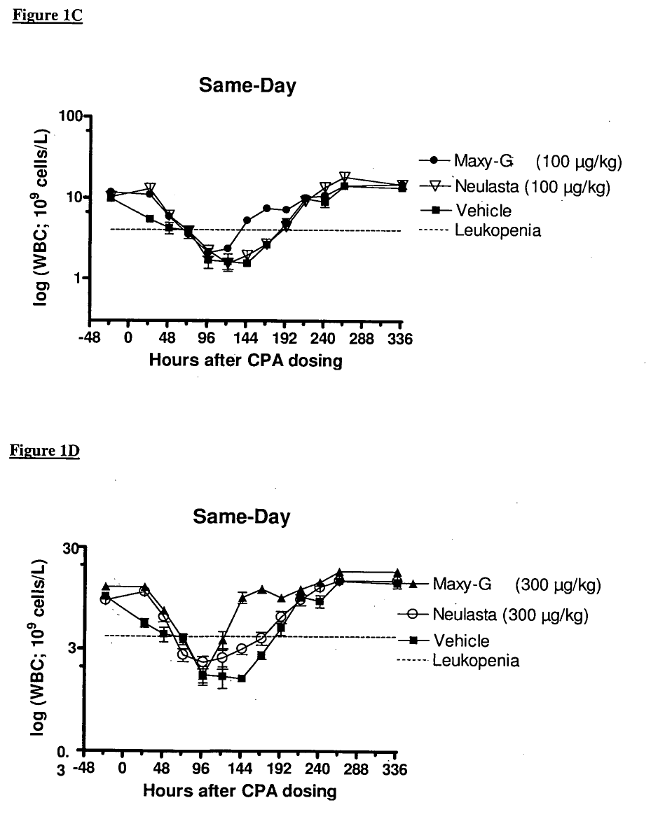

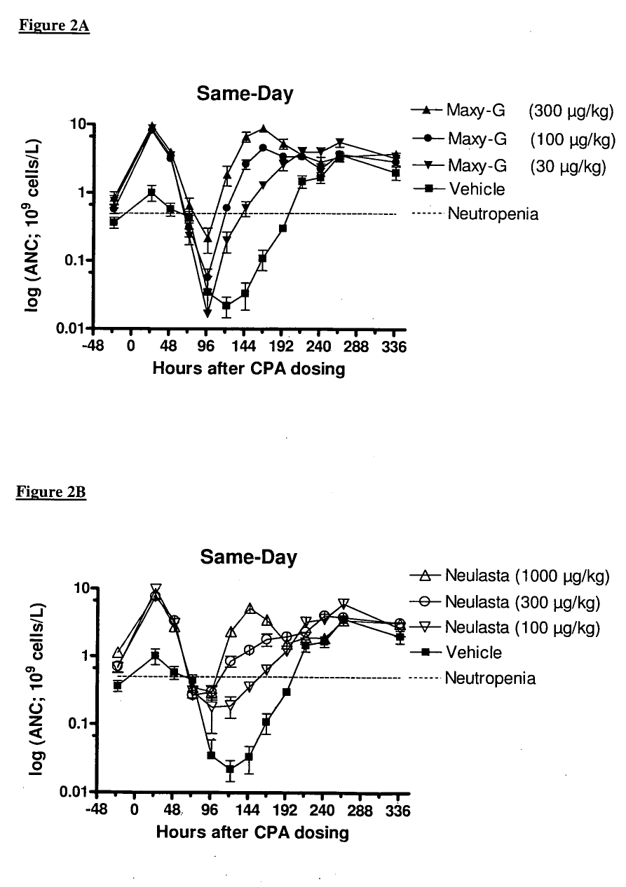

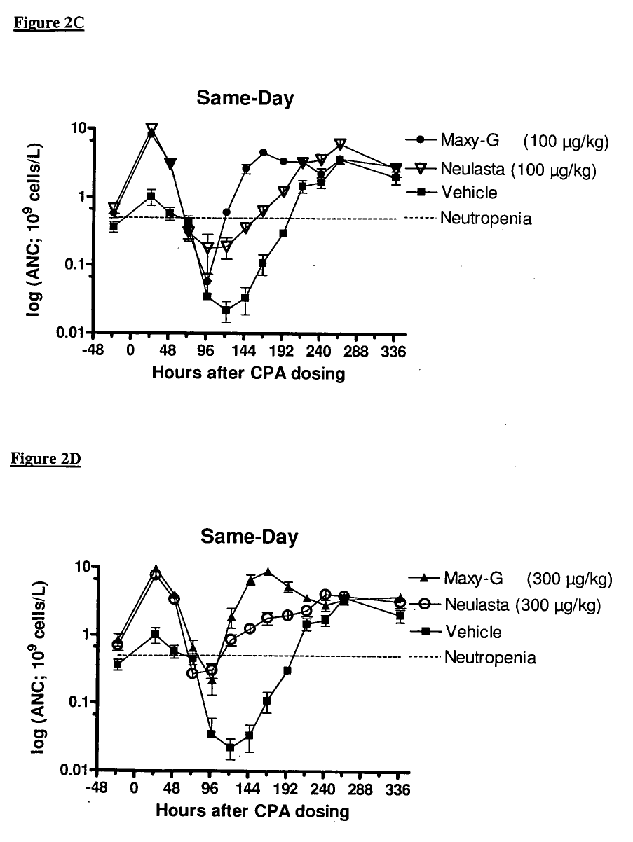

This data is represented graphically in FIGS. 1 and 2, in which the effects of Maxy-G and Neulasta® on the hematological responses to CPA-treatment are displayed. Each data point represents the mean±standard error of the mean (SEM) of six individual animals. In FIG. 1, the dose response relationship of Maxy-G (FIG. 1A) and Neulasta® (FIG. 1B) on CPA-induced leukopenia is shown. FIGS. 1C and 1D compare the effects of equivalent doses (in μg/kg) of the two G-CSF conjugates on CPA-induced leukopenia (WBC counts), demonstrating the larger effect of Maxy-G in this model. FIG. 2 presents the effect of vehicle, Maxy-G, and Neulasta on the ANC levels on CPA-induced neutropenia (i.e. dose-response data for Maxy-G and Neulasta® in FIGS. 2A and 2B followed by comparative data at two dose-equivalent doses of Maxy-G and Neulasta® in FIGS. 2C and 2D).

Pharmacokinetics: After subcutaneous administration of Maxy-G at dose levels of 30, 100 and 300 μg/kg, good systemic exposure and distribution are observed allowing estimates of Tₘₐₓ (time to maximum concentration), Cₘₐₓ (maximum concentration), and AUC₀₋ₜ (area under the curve from time 0 to the last data point) to be made (Table 3). The Tₘₐₓ values for both compounds are observed at 24 h for animals in all dose groups (the first sampling point after subcutaneous dosing).

In terms of relative systemic exposure between the two compounds, it is clear from Table 3 that AUC₀₋ₜ values are higher for Maxy-G than for Neulasta® at the same dose level. Specifically, at 100 μg/kg, the AUC₀₋ₜ for Maxy-G is approximately 2.6-fold higher than for Neulasta® and about 2-fold higher at the 300 μg/kg dose level. The Cₘₐₓ values show a similar increase, albeit not as high, being on average approximately 1.6-fold higher for Maxy-G than for Neulasta® at the same two dose levels.

TABLE 3
Comparative pharmacokinetic data following subcutaneous
administration of Maxy-G or Neulasta ® to neutropenic rats.

| | | | C_{max (ng/mL)} | AUC₀₋ₜ (ng.h/mL) |
|---|---|---|---|---|
| Compound | Dose (μg/kg) | Tₘₐₓ (h) | Mean ± SD | Mean ± SD |
| Maxy-G | 30 | 24 | 48 ± 7 | 2116 ± 250 |
| Maxy-G | 100 | 24 | 280 ± 37 | 14534 ± 2308 |
| Maxy-G | 300 | 24 | 951 ± 275 | 51491 ± 13203 |
| Neulasta ® | 100 | 24 | 163 ± 29 | 5510 ± 1193 |
| Neulasta ® | 300 | 24 | 638 ± 105 | 26323 ± 4272 |
| Neulasta ® | 1000 | 24 | 2922 ± 333 | 143860 ± 12365 |

### Discussion and Conclusion

Treatment of normopenic rats with cyclophosphamide results in myelosuppression, which is evidenced by profound and prolonged neutropenia and leukopenia. This effect provides a convenient neutropenic experiment model for the characterization of therapies designed to stimulate WBC and ANC production. In this model, with a same-day administration protocol with cyclophosphamide, Maxy-G is particularly effective. While both drugs demonstrate dose-dependency in both of these parameters, Maxy-G appears to be more effective in this model, as 30 μg/kg Maxy-G is approximately as effective as 100 μg/kg Neulasta®.

Overall, the systemic exposure, on an equal dose basis, not only demonstrates that Maxy-G yields significantly higher values for both Cₘₐₓ and AUC₀₋ₜ than for Neulasta®, but also indicates that the systemic levels of Maxy-G are sustained for a longer time period. Specifically, at the 100 and 300 μg/kg dose levels, Neulasta® is systemically cleared by about 96 h, while the exposure time for Maxy-G is considerably longer; about 168 h.

The present results with same-day administration of G-CSF thus suggest that:
Maxy-G shortens the time-to-recovery (TTR) and the time of neutropenia/leukopenia in a dose-dependent manner in CPA-treated rats;
Maxy-G is as effective as an about 3-fold higher dose of Neulasta®; and
Maxy-G has an approximately 3-fold higher AUC₀₋ₜ (at 100 μg/kg) than Neulasta®.
   It is concluded that Maxy-G is effective in a same-day administration protocol with cyclophosphamide.

### Example 3

### Same-Day Administration of PEGylated G-CSF and Paclitaxel

A pilot study was performed to evaluate the ability of a multi-PEGylated G-CSF variant to counteract or minimize the period of neutropenia induced by Paclitaxel, a chemotherapeutic agent, in male rats, and to compare the effect to that of the mono-PEGylated G-CSF, Neulasta®.

### Materials and Methods

Chemotherapeutic agent: Paclitaxel (Taxol, 6 mg/mL)

Reference mono-PEGylated G-CSF: Neulasta® (see Example 2)

Test multi-PEGylated G-CSF variant: “Maxy-G” (see Example 2)

Vehicle: 10 mM sodium acetate, pH 4.0, 45 mg/mL mannitol in water for injection,

0.05 mg/mL Tween 20.

Animals: Sprague-Dawley rats; age at initiation of treatment: approximately 6 weeks; approximate body weight range at initiation of treatment: 170-220 g; pelleted complete diet and water ad libitu m; main group: 50 males; satellite animals for bioanalysis: 30 males.

Experimental design: On day 0, the animals were treated with the chemotherapy agent (Paclitaxel) in a dose of 6 mg/kg (10 mL/kg of the agent in a dose concentration of 0.6 mg/mL). The chemotherapy agent was administered by intravenous injection at a rate of 1 mL/kg/minute. Two hours later, the animals were treated with the PEGylated G-CSF molecules (Maxy-G or Neulasta®) or vehicle as follows:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1. | vehicle | 0 | 1 | 0 | 10 | 6 |
| 2. | Maxy-G | 0.1 | 1 | 0.1 | 10 | 6 |
| 3. | Maxy-G | 0.3 | 1 | 0.3 | 10 | 6 |
| 4. | Neulasta ® | 0.1 | 1 | 0.1 | 10 | 6 |
| 5. | Neulasta ® | 0.3 | 1 | 0.3 | 10 | 6 |

The dose level of 0.3 mg/kg for Maxy-G is equivalent to an average proposed human clinical dose of 50 μg/kg, based on relative body surface area. The G-CSF conjugates were administered subcutaneously by bolus injection. Blood samples were collected from three animals per group for analysis of the neutrophil count at 6, 12, 24, 36, 48, 96, 120, 144 and 192 hours after administration of G-CSF conjugate or vehicle.

### Results

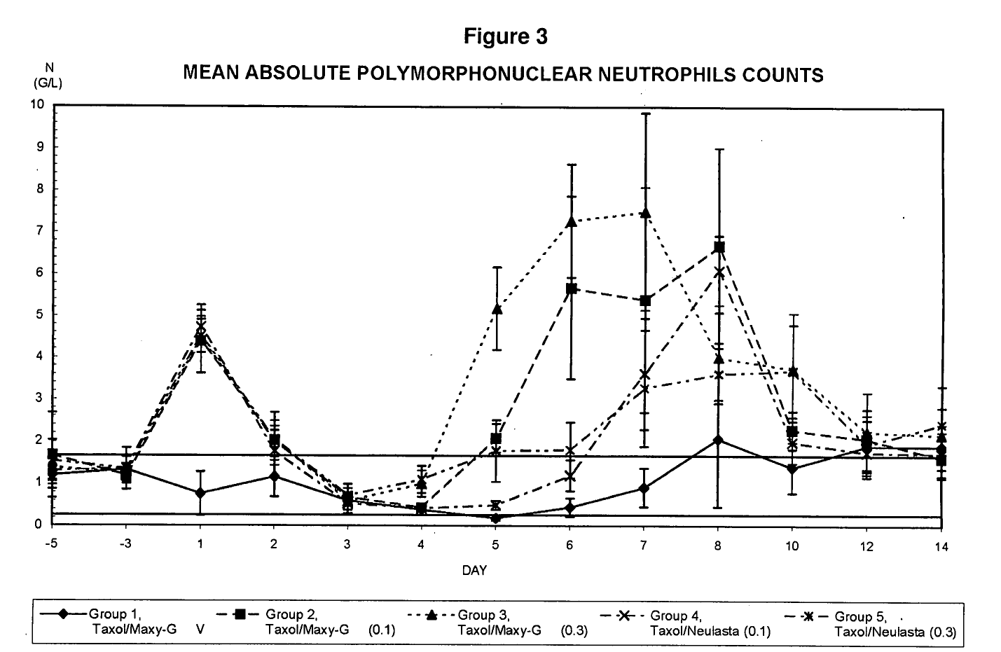

The data for the neutrophil counts in this study demonstrates that same-day administration of either 0.1 or 0.3 mg/kg of Maxy-G according to the invention to counteract Paclitaxel-induced neutropenia provides an improved neutrophil stimulation and a reduced duration of neutropenia compared to equivalent doses of Neulasta®. The neutrophil count data are shown graphically in FIG. 3, in which the two horizontal lines indicate approximate levels for low normal and high normal neutrophil counts. The figure shows that same-day administration of either dose of Maxy-G or Neulasta® provides a substantially improved recovery of the neutrophil count compared to the control group (1) that received only the vehicle, and that there is a clear tendency towards a faster and more pronounced effect in groups 2 and 3 that received Maxy-G compared to groups 4 and 5 that received Neulasta®.

### Conclusion

The data demonstrate that same-day administration of Maxy-G is effective at reducing Paclitaxel-induced neutropenia. Furthermore the data also suggest that Maxy-G is more effective in this regard than administration of an equivalent dose of Neulasta®.

### Example 4

### Same-Day Administration Studies of a Multi-PEGylated G-CSF Variant with Various Chemotherapeutic Agents

Since G-CSF recruits dormant cells into the cell cycle, administration of a G-CSF molecule on the same day as administration of a cytotoxic (chemotherapeutic) agent could conceivably potentiate chemotherapy-induced myelosuppression. Pre-clinical studies were conducted to evaluate the effect of Maxy-G on chemotherapy-induced neutropenia in rats when Maxy-G is administered either 2 hours or 24 hours after chemotherapy treatment. A variety of chemotherapeutic agents were tested, which are representative of a wide range of classes of chemotherapeutic agents currently in clinical use:

A. Doxorubicin, an antitumor antibiotic/Topoisomerase II inhibitor and DNA intercalator, which is commonly used in the treatment of solid tumors and hematologic malignancies;

B. Carboplatin, an inorganic metal complex agent/alkylating agent and DNA synthesis inhibitor, which is commonly used in the treatment of testicular carcinoma, breast, ovarian and bladder cancers;

C. Cyclophosphamide, an alkylating agent, which is commonly used in the treatment of non-Hodgkin's lymphoma, leukemias, multiple myeloma, breast & ovarian cancers; and

D. Vincristine, a vinca alkaloid/antimicrotubule agent, which is commonly used in the treatment of Hodgkin's and non-Hodgkin's lymphomas and multiple myeloma.

These studies were designed to approximate human dosing conditions and routes of administration. The dose level of the chemotherapeutic agents used in these studies were each selected based on prior studies in order to provide between a 30% and 70% reduction in ANC in the absence of G-CSF. An ANC reduction in this range (in the absence of G-CSF) should permit the detection of either potential adverse or beneficial effects of Maxy-G administration on the ANC profile. The dose of Maxy-G used in these studies was equivalent to a proposed human clinical dose of approximately 50 μg/kg, based on body surface area.

A. ANC Profiles for Same-Day Vs. Next-Day Administration of Maxy-G Plus Doxorubicin

### Materials and Methods

Chemotherapeutic agent: Doxorubicin (Adriamycin), 2 mg/mL.

Multi-PEGylated G-CSF variant: “Maxy-G” (see Example 2); 10.3 mg/mL solution in vehicle.

Vehicle: 10 mM sodium acetate, pH 4.0, 45 mg/mL mannitol in water for injection, 0.05 mg/mL Tween 20.

Animals: Sprague-Dawley rats; age at initiation of treatment: approximately 6 weeks; approximate body weight range at initiation of treatment: 170-220 g; pelleted complete diet and water ad libitum; Number of animals in the study: 105 males; main group: 60 males; satellite animals for bioanalysis: 40 males, animals for baseline data: 5 males. Animals were acclimatized for seven days minimum between arrival and start of treatment.

Experimental design: The study was performed as outlined below.

| Group | Day 0 (IV) | Day 0 (SC) | Day 1 (SC) |
|---|---|---|---|
| 1 | SPS | Vehicle (+2 hours) | — |
| 2 | Doxorubicin | Vehicle (+2 hours) | — |
| 3 | Doxorubicin | Maxy-G (+2 hours) | — |
| 4 | Doxorubicin | — | Maxy-G (+24 hours) |

IV: intravenous administration
SC: subcutaneous administration
SPS: sterile physiological saline

On day 0, the animals were treated with the chemotherapy agent (Doxorubicin) at a dose of 4 mg/kg (5 mL/kg of the agent at a dose concentration of 0.8 mg/mL in SPS) or SPS alone. The chemotherapy agent or SPS was administered by intravenous injection at a rate of 1 mL/minute according to the following schedule:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1 | SPS | 0 | 5 | 0 | — | 10 |
| 2 | Doxorubicin | 4 | 5 | 0.8 | 20 | 10 |
| 3 | Doxorubicin | 4 | 5 | 0.8 | 20 | 10 |
| 4 | Doxorubicin | 4 | 5 | 0.8 | 20 | 10 |

Individual dose volumes were calculated using the body weight recorded on day 0. Doxorubicin or SPS was administered intravenously into a tail vein.

Two or twenty-four hours later, as indicated, the animals were treated with Maxy-G or vehicle as follows:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1 | Vehicle (+2 hrs) | 0 | 1 | 0 | — | 10 |
| 2 | Vehicle (+2 hrs) | 0 | 1 | 0 | 20 | 10 |
| 3 | Maxy-G (+2 hrs) | 0.3 | 1 | 0.3 | 20 | 10 |
| 4 | Maxy-G (+24 hrs) | 0.3 | 1 | 0.3 | 20 | 10 |

Individual dose volumes were calculated using the body weight recorded on day 0 for Groups 1, 2 and 3 (administration of Maxy-G or vehicle two hours after Doxorubicin) and on day 1 for Group 4 (administration of Maxy-G twenty-four hours after Doxorubicin). Maxy-G or vehicle was administered subcutaneously by bolus injection. Blood samples (0.5 ml maximum) were withdrawn from the sublingual vein following isoflurane anesthesia of unfasted animals, for analysis of neutrophil counts. These samples were withdrawn from five animals per satellite group on days 1, 3, 5, 7, and 9, and from the remaining five animals per satellite group on days 2, 4, 6, 8, 10, and 14.

### Results and Conclusion

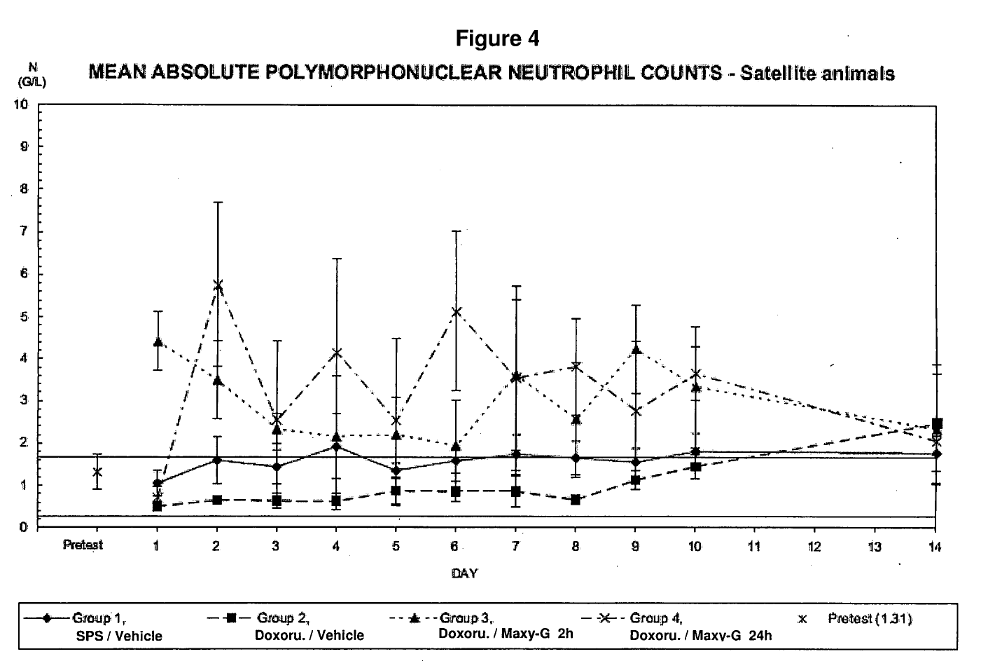

The ANC profiles for 2 hour vs. 24 hour administration of Maxy-G following administration of Doxorubicin are shown graphically in FIG. 4. The two horizontal lines indicate approximate levels for low normal and high normal neutrophil counts. The figure shows that administration of Maxy-G two hours after administration of Doxorubicin counteracted the myelosuppressive effect of Doxorubicin. The data obtained in this study thus demonstrates that no aggravated myelosuppression is observed following same-day administration of Maxy-G and the chemotherapeutic agent Doxorubicin, and supports the use of Maxy-G in a same-day administration protocol with Doxorubicin.

B. ANC Profiles for Same-Day Vs. Next-Day Administration of Maxy-G Plus Carboplatin

### Materials and Methods

Chemotherapeutic agent: Carboplatin (platinum coordination complex).

Multi-PEGylated G-CSF variant: “Maxy-G” (see Example 2); 10.3 mg/mL solution in vehicle.

Vehicle: 10 mM sodium acetate, pH 4.0, 45 mg/mL mannitol in water for injection, 0.05 mg/mL Tween 20.

Animals: Sprague-Dawley rats; age at initiation of treatment: approximately 6 weeks; approximate body weight range at initiation of treatment: 170-220 g; pelleted complete diet and water ad libitum; Number of animals in the study: 100 males; main group: 60 males; satellite animals for bioanalysis: 40 males. Animals were acclimatized for seven days minimum between arrival and start of treatment.

Experimental design: The study was performed as outlined below.

| Group | Day 0 (IV) | Day 0 (SC) | Day 1 (SC) |
|---|---|---|---|
| 1 | SPS | Vehicle (+2 hours) | — |
| 2 | Carboplatin | Vehicle (+2 hours) | — |
| 3 | Carboplatin | Maxy-G (+2 hours) | — |
| 4 | Carboplatin | — | Maxy-G (+24 hours) |

IV: intravenous administration
SC: subcutaneous administration
SPS: sterile physiological saline

On day 0, the animals were treated with the chemotherapy agent (Carboplatin) at a dose of 40 mg/kg (5 mL/kg of the agent at a dose concentration of 8 mg/mL in SPS) or SPS alone. The chemotherapy agent or SPS was administered by intravenous injection at a rate of 1 mL/minute according to the following schedule:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1 | SPS | 0 | 5 | 0 | — | 10 |
| 2 | Carboplatin | 40 | 5 | 8 | 20 | 10 |
| 3 | Carboplatin | 40 | 5 | 8 | 20 | 10 |
| 4 | Carboplatin | 40 | 5 | 8 | 20 | 10 |

Individual dose volumes were calculated using the body weight recorded on day 0. Carboplatin or SPS was administered intravenously into a tail vein.

Two or twenty-four hours later, as indicated, the animals were treated with Maxy-G or vehicle as follows:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1 | Vehicle (+2 hrs) | 0 | 1 | 0 | — | 10 |
| 2 | Vehicle (+2 hrs) | 0 | 1 | 0 | 20 | 10 |
| 3 | Maxy-G (+2 hrs) | 0.3 | 1 | 0.3 | 20 | 10 |
| 4 | Maxy-G (+24 hrs) | 0.3 | 1 | 0.3 | 20 | 10 |

Individual dose volumes were calculated using the body weight recorded on day 0 for Groups 1, 2 and 3 (administration of Maxy-G or vehicle two hours after Carboplatin) and on day 1 for Group 4 (administration of Maxy-G twenty-four hours after Carboplatin). Maxy-G or vehicle was administered subcutaneously by bolus injection. Blood samples (0.5 ml maximum) were withdrawn from the sublingual vein following isoflurane anesthesia of unfasted animals, for analysis of neutrophil counts. These samples were withdrawn from five animals per satellite group on days 1, 3, 5, 7, and 9, and from the remaining five animals per satellite group on days 2, 4, 6, 8, 10, and 14.

### Results and Conclusion

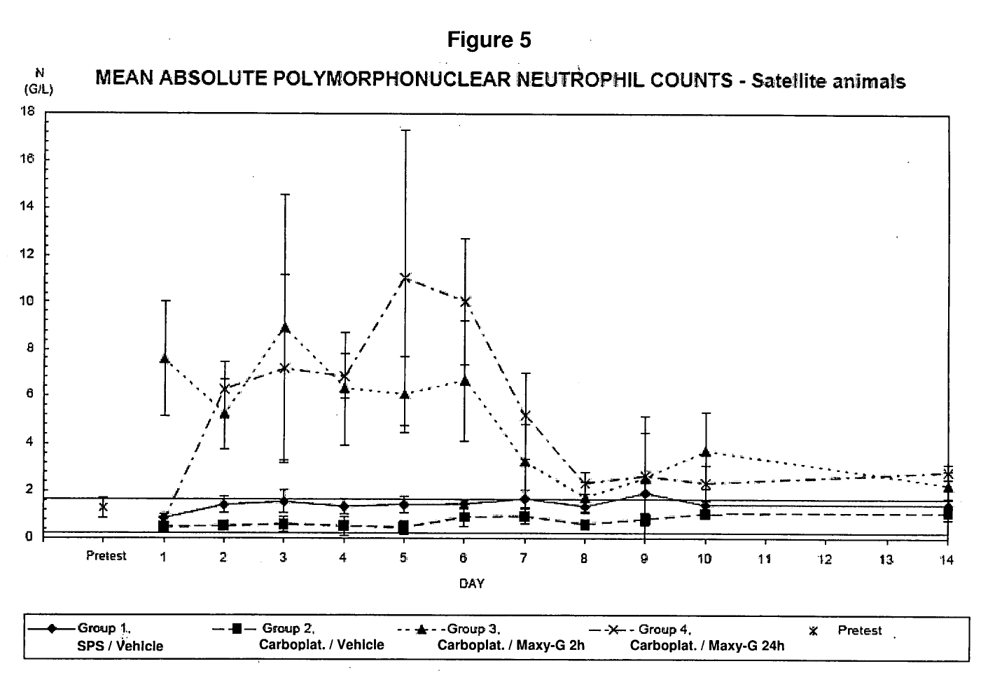

The ANC profiles for 2 hour vs. 24 hour administration of Maxy-G following administration of Carboplatin are shown graphically in FIG. 5. The two horizontal lines indicate approximate levels for low normal and high normal neutrophil counts. The figure shows that administration of Maxy-G two hours after Carboplatin administration counteracted the myelosuppressive effect of Carboplatin. The data obtained in this study thus demonstrates that no aggravated myelosuppression is observed following same-day administration of Maxy-G and the chemotherapeutic agent Carboplatin, and supports the use of Maxy-G in a same-day administration protocol with Carboplatin.

C. ANC Profiles for Same-Day Vs. Next-Day Administration of Maxy-G Plus Cyclophosphamide

### Materials and Methods

Chemotherapeutic agent: Cyclophosphamide (Sendoxan 1000 mg).

Multi-PEGylated G-CSF variant: “Maxy-G” (see Example 2); 10.3 mg/mL solution in vehicle.

Vehicle: 10 mM sodium acetate, pH 4.0, 45 mg/mL mannitol in water for injection, 0.05 mg/mL Tween 20.

Animals: Sprague-Dawley rats; age at initiation of treatment: approximately 6 weeks; approximate body weight range at initiation of treatment: 170-220 g; pelleted complete diet and water ad libitum; main group: 50 males; satellite animals for bioanalysis: 40 males; animals for baseline data: 5 males. Animals were acclimatized for seven days minimum between arrival and start of treatment.

Experimental design: The study was performed as outlined below.

| Group | Day 0 (IV) | Day 0 (SC) | Day 1 (SC) |
|---|---|---|---|
| 1 | SPS | Vehicle (+2 hours) | — |
| 2 | Cyclophosphamide | Vehicle (+2 hours) | — |
| 3 | Cyclophosphamide | Maxy-G (+2 hours) | — |
| 4 | Cyclophosphamide | — | Maxy-G (+24 hours) |

IV: intravenous administration
SC: subcutaneous administration
SPS: sterile physiological saline

On day 0, the animals were treated with the chemotherapy agent (Cyclophosphamide) at a dose of 20 mg/kg (5 mL/kg of the agent at a dose concentration of 4 mg/mL in SPS) or SPS alone. The chemotherapy agent or SPS was administered by intravenous injection at a rate of 1 mL/minute according to the following schedule:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1 | SPS | 0 | 5 | 0 | — | 10 |
| 2 | Cyclophosphamide | 20 | 5 | 4 | 20 | 10 |
| 3 | Cyclophosphamide | 20 | 5 | 4 | 20 | 10 |
| 4 | Cyclophosphamide | 20 | 5 | 4 | 20 | 10 |

Individual dose volumes were calculated using the body weight recorded on day 0. Cyclophosphamide or SPS was administered intravenously into a tail vein.

Two or twenty-four hours later, as indicated, the animals were treated with Maxy-G or vehicle as follows:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1 | Vehicle (+2 hrs) | 0 | 1 | 0 | — | 10 |
| 2 | Vehicle (+2 hrs) | 0 | 1 | 0 | 20 | 10 |
| 3 | Maxy-G (+2 hrs) | 0.3 | 1 | 0.3 | 20 | 10 |
| 4 | Maxy-G (+24 hrs) | 0.3 | 1 | 0.3 | 20 | 10 |

Individual dose volumes were calculated using the body weight recorded-on day 0 for Groups 1, 2 and 3 (administration of Maxy-G or vehicle two hours after Cyclophosphamide) and on day 1 for Group 4 (administration of Maxy-G twenty-four hours after Cyclophosphamide). Maxy-G or vehicle was administered subcutaneously by bolus injection. Blood samples (0.5 ml maximum) were withdrawn from the sublingual vein following isoflurane anesthesia of unfasted animals, for analysis of neutrophil counts. These samples were withdrawn from five animals per satellite group on days 1, 3, 5, 7, and 9, and from the remaining five animals per satellite group on days 2, 4, 6, 8, 10, and 14.

### Results and Conclusion

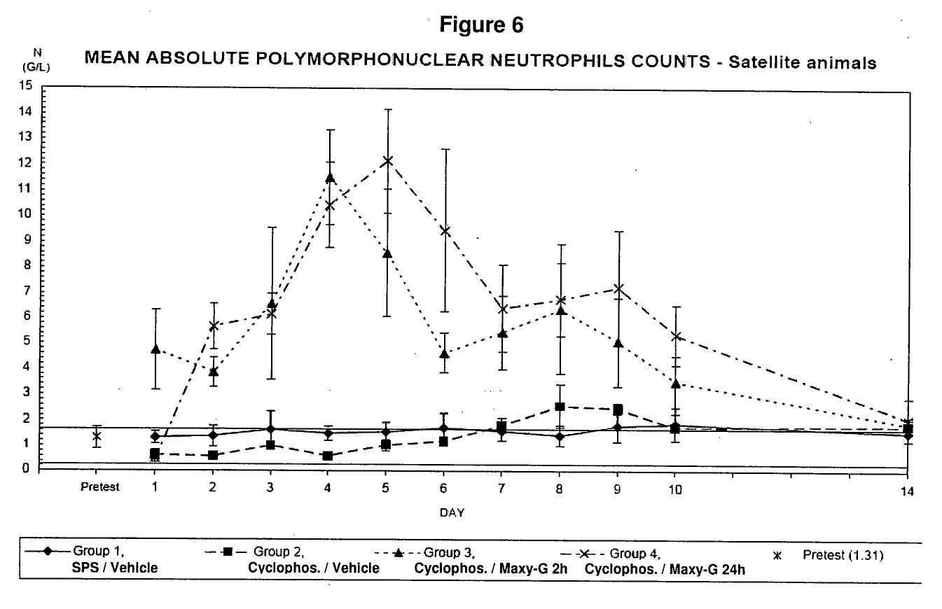

The ANC profiles for 2 hour vs. 24 hour administration of Maxy-G following administration of Cyclophosphamide are shown graphically in FIG. 6. The two horizontal lines indicate approximate levels for low normal and high normal neutrophil counts. The figure shows that administration of Maxy-G two hours after administration of Cyclophosphamide counteracted the myelosuppressive effect of Cyclophosphamide. The data obtained in this study thus demonstrates that no aggravated myelosuppression is observed following same-day administration of Maxy-G and the chemotherapeutic agent Cyclophosphamide, and supports the use of Maxy-G in a same-day administration protocol with Cyclophosphamide.

D. ANC Profiles for Same-Day Vs. Next-Day Administration of Maxy-G Plus Vincristine

### Materials and Methods

Chemotherapeutic agent: Vincristine (Vincristine sulphate), 1 mg/mL.

Multi-PEGylated G-CSF variant: “Maxy-G” (see Example 2); 10.3 mg/mL solution in vehicle.

Vehicle: 10 mM sodium acetate, pH 4.0, 45 mg/mL mannitol in water for injection, 0.05 mg/mL Tween 20.

Animals: Sprague-Dawley rats; age at initiation of treatment: approximately 6 weeks; approximate body weight range at initiation of treatment: 170-220 g; pelleted complete diet and water ad libitum; Number of animals in the study: 100 males; main group: 60 males; satellite animals for bioanalysis: 40 males. Animals were acclimatized for seven days minimum between arrival and start of treatment.

Experimental design: The study was performed as outlined below.

| Group | Day 0 (IV) | Day 0 (SC) | Day 1 (SC) |
|---|---|---|---|
| 1 | SPS | Vehicle (+2 hours) | — |
| 2 | Vincristine | Vehicle (+2 hours) | — |
| 3 | Vincristine | Maxy-G (+2 hours) | — |
| 4 | Vincristine | — | Maxy-G (+24 hours) |

IV: intravenous administration
SC: subcutaneous administration
SPS: sterile physiological saline

On day 0, the animals were treated with the chemotherapy agent (Vincristine) at a dose of 0.15 mg/kg (5 mL/kg of the agent at a dose concentration of 0.03 mg/mL in SPS) or SPS alone. The chemotherapy agent or SPS was administered by intravenous injection at a rate of 1 mL/minute according to the following schedule:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1 | SPS | 0 | 5 | 0 | — | 10 |
| 2 | Vincristine | 0.15 | 5 | 0.03 | 20 | 10 |
| 3 | Vincristine | 0.15 | 5 | 0.03 | 20 | 10 |
| 4 | Vincristine | 0.15 | 5 | 0.03 | 20 | 10 |

Individual dose volumes were calculated using the body weight recorded on day 0. Vincristine or SPS was administered intravenously into a tail vein.

Two or twenty-four hours later, as indicated, the animals were treated with Maxy-G or vehicle as follows:

| | | | | Dose | Number of animals | |
|---|---|---|---|---|---|---|
| | | Dose level | Dose volume | concentration | Main group | Satellite |
| Group | Treatment | (mg/kg) | (mL/kg) | (mg/mL) | males | males |
| 1 | Vehicle (+2 hrs) | 0 | 1 | 0 | — | 10 |
| 2 | Vehicle (+2 hrs) | 0 | 1 | 0 | 20 | 10 |
| 3 | Maxy-G (+2 hrs) | 0.3 | 1 | 0.3 | 20 | 10 |
| 4 | Maxy-G (+24 hrs) | 0.3 | 1 | 0.3 | 20 | 10 |

Individual dose volumes were calculated using the body weight recorded on day 0 for Groups 1, 2 and 3 (administration of Maxy-G or vehicle two hours after Vincristine) and on day 1 for Group 4 (administration of Maxy-G twenty-four hours after Vincristine). Maxy-G or vehicle was administered subcutaneously by bolus injection. Blood samples (0.5 ml maximum) were withdrawn from the sublingual vein following isoflurane anesthesia of unfasted animals, for analysis of neutrophil counts. These samples were withdrawn from five animals per satellite group on days 1, 3, 5, 7, and 9, and from the remaining five animals per satellite group on days 2, 4, 6, 8, 10, and 14.

### Results and Conclusion

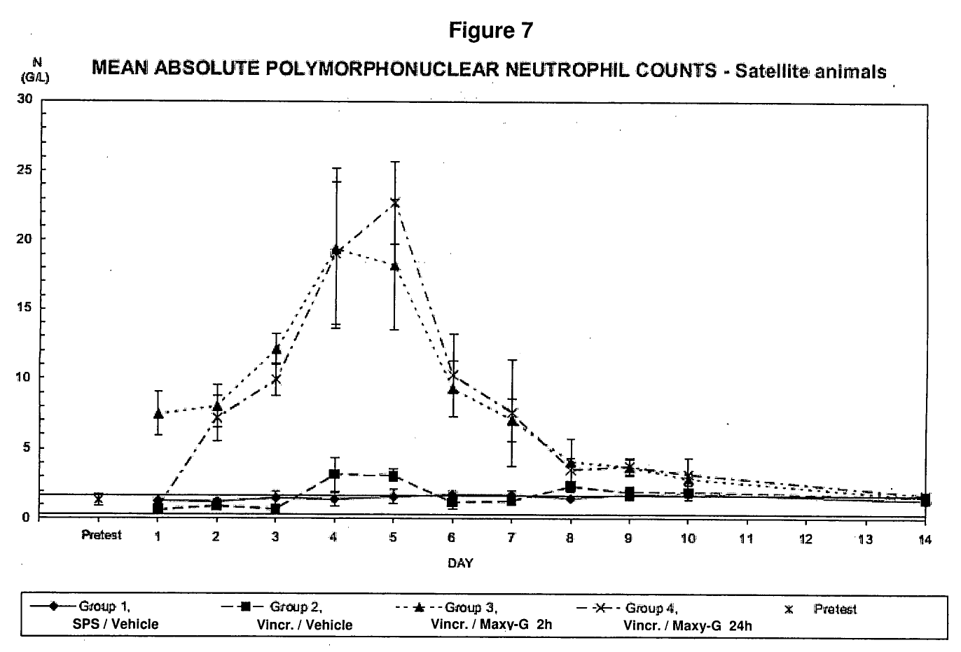

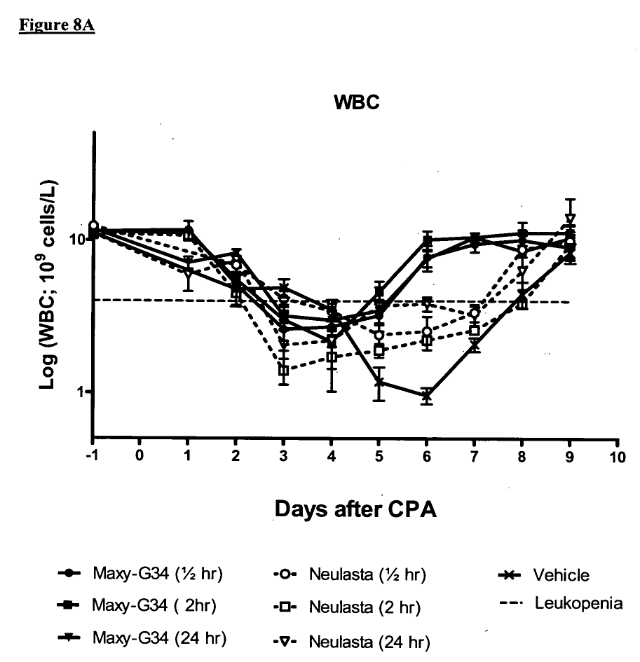

The ANC profiles for 2 hour vs. 24 hour administration of Maxy-G following administration of Vincristine are shown graphically in FIG. 7. The two horizontal lines indicate approximate levels for low normal and high normal neutrophil counts. The figure shows that administration of Maxy-G two hours after administration of Vincristine counteracted the myelosuppressive effect of Vincristine. The data obtained in this study thus demonstrates that no aggravated myelosuppression is observed following same-day administration of Maxy-G and the chemotherapeutic agent Vincristine, and supports the use of Maxy-G in a same-day administration protocol with Vincristine.

### Example 5

### Same-Day Administration of Chemotherapy and Maxy-G in the Treatment of Breast Cancer

### TAC Regimen:

A patient with breast cancer is treated with a combination chemotherapy regimen of docetaxel, doxorubicin, and cyclophosphamide (“TAC regimen”). Multi-PEGylated G-CSF variant Maxy-G is administered the same day as the completion of administration of the chemotherapeutic agents.

The patient is administered a cycle of chemotherapy in which 75 mg/m² docetaxel, 50 mg/m² doxorubicin, and 500 mg/m² cyclophosphamide are administered intravenously on day 1 according to standard clinical practice. The exact dosage of each drug depends on a number of factors, such as the weight, age and the severity of the disease, and may be ascertained by those of skill in the art. Within about two to six hours after completion of the administration of chemotherapy, 3-12 mg (or alternatively, 50 μg/kg to 150 μg/kg) of Maxy-G is administered to the patient subcutaneously. This cycle of chemotherapy followed by Maxy-G is repeated every 14-21 days for three to five cycles.

### Doxorubicin Plus Cyclophosphamide (AC Regimen):

A patient with breast cancer is treated with a combination chemotherapy regimen of doxorubicin plus cyclophosphamide (“AC regimen”). Multi-PEGylated G-CSF variant Maxy-G is administered the same day as the completion of administration of the chemotherapeutic agents.

The patient is administered a cycle of chemotherapy in which 60 mg/m² doxorubicin and 600 mg/m² cyclophosphamide are administered intravenously on day 1 according to standard clinical practice. The exact dosage of each drug depends on a number of factors, such as the weight, age and the severity of the disease, and may be ascertained by those of skill in the art. Within about two to six hours after completion of the administration of chemotherapy, 3-12 mg (or alternatively, 50 μg/kg to 150 μg/kg) of Maxy-G is administered to the patient subcutaneously. This cycle of chemotherapy followed by Maxy-G is repeated every 14-21 days for three to five cycles.

### Doxorubicin Plus Cyclophosphamide, Followed by Paclitaxel (AC+P Regimen):

A patient with breast cancer is treated with a combination chemotherapy regimen of doxorubicin plus cyclophosphamide, followed by a second series of chemotherapy employing paclitaxel (“AC+P regimen”). Multi-PEGylated G-CSF variant Maxy-G is administered the same day as the completion of administration of the chemotherapeutic agents.

The patient is administered an initial cycle of chemotherapy in which 60 mg/m² doxorubicin and 600 mg/m² cyclophosphamide are administered intravenously on day 1 according to standard clinical practice. The exact dosage of each drug depends on a number of factors, such as the weight, age and the severity of the disease, and may be ascertained by those of skill in the art. Within about two to six hours after completion of the administration of chemotherapy, 3-12 mg (or alternatively, 50 μg/kg to 150 μg/kg) of Maxy-G is administered to the patient subcutaneously. This cycle of chemotherapy followed by Maxy-G is repeated every 21 days for four cycles.

Twenty-one days following the fourth cycle of doxorubicin/cyclophosphamide/Maxy-G, a new cycle of chemotherapy is initiated in which the patient is administered 175 mg/m² paclitaxel intravenously according to standard clinical practice. The exact dosage of this drug depends on a number of factors, such as the weight, age and the severity of the disease, and may be ascertained by those of skill in the art. Within about two to six hours after completion of paclitaxel administration, 3-12 mg (or alternatively, 50 μg/kg to 150 μg/kg) of Maxy-G is administered to the patient subcutaneously. This cycle of paclitaxel followed by Maxy-G is repeated every 21 days for four cycles.

### Example 6

### Same-Day Administration of Chemotherapy and Maxy-G in the Treatment of Lung Cancer

### Cisplatin Plus Etoposide (EP Regimen):

A patient with small cell lung cancer is treated with a combination chemotherapy regimen of etoposide plus cisplatin (EP regimen). Multi-PEGylated G-CSF variant Maxy-G is administered the same day as the completion of administration of the chemotherapeutic agents.

The patient is administered a cycle of chemotherapy in which 60-80 mg/m² cisplatin is administered intravenously on day 1, and 80-120 mg/m² etoposide is administered intravenously on days 1-3, according to standard clinical practice. The exact dosage of each drug depends on a number of factors, such as the weight, age and the severity of the disease, and may be ascertained by those of skill in the art. Within about two to six hours after completion of the administration of etoposide on day 3, 3-12 mg (or alternatively, 50 μg/kg to 150 μg/kg) of Maxy-G is administered to the patient subcutaneously. This cycle of chemotherapy followed by Maxy-G is repeated every 21-28 days for three to five cycles.

### Carboplatin Plus Paclitaxel:

A patient with non-small cell lung cancer is treated with a combination chemotherapy regimen of carboplatin plus paclitaxel. Multi-PEGylated G-CSF variant Maxy-G is administered the same day as the completion of chemotherapy in each cycle.

The patient is administered a cycle of chemotherapy in which 175-225 mg/m² paclitaxel is administered intravenously on day 1 over a period of about three hours, followed by carboplatin which is administered intravenously on day 1 to an AUC of 5-6, according to standard clinical practice. The exact dosage of each drug depends on a number of factors, such as the weight, age and the severity of the disease, and may be ascertained by those of skill in the art. Within about two to six hours after completion of the administration of chemotherapy, 3-12 mg (or alternatively, 50 μg/kg to 150 μg/kg) of Maxy-G is administered to the patient subcutaneously. This cycle of chemotherapy followed by Maxy-G is repeated every 21 days for three to five cycles.

### Example 7

### Same-Day Administration of Chemotherapy and Maxy-G in the Treatment of Non-Hodgkin's Lymphoma

### Cyclophosphamide, Doxorubicin, Vincristine and Rituximab (CHOP-R Regimen):

A patient with Non-Hodgkin's Lymphoma (NHL) is treated with a combination chemotherapy regimen of cyclophosphamide, doxorubicin, vincristine, and rituximab, plus prednisone (“CHOP-R regimen”). Multi-PEGylated G-CSF variant Maxy-G is administered the same day as the completion of the administration of the chemotherapeutic agents in each cycle.

The patient is administered a cycle of chemotherapy in which 375 mg/m² rituximab is administered intravenously on day 1, which is followed by 750 mg/m² cyclophosphamide, 50 mg/m² doxorubicin, and 1.4 mg/m² vincristine (maximum, 2 mg) administered intravenously on day 1, according to standard clinical practice. The exact dosage of each drug depends on a number of factors, such as the weight, age and the severity of the disease, and may be ascertained by those of skill in the art. Within about two to six hours after completion of chemotherapy, 3-12 mg (or alternatively, 50 μg/kg to 150 μg/kg) of Maxy-G is administered to the patient subcutaneously. In addition, 40 mg/m² of prednisone, an anti-inflammatory corticosteroid, is administered PO on days 1-5. This cycle of chemotherapy followed by Maxy-G and prednisone is repeated every 21 days for three to five cycles.

Alternatively, the patient is administered a cycle of chemotherapy in which 375 mg/m² rituximab is administered intravenously on day 1 according to standard clinical practice, followed on day 3 by 750 mg/m² cyclophosphamide, 50 mg/m² doxorubicin, and 1.4 mg/m² vincristine (maximum, 2 mg) administered intravenously according to standard clinical practice. The exact dosage of each drug depends on a number of factors, such as the weight, age and the severity of the disease, and may be ascertained by those of skill in the art. Within about two to six hours after completion of the administration of the cyclophosphamide, doxorubicin, and vincristine on day 3, 3-12 mg (or alternatively, 50 μg/kg to 150 μg/kg) of Maxy-G is administered to the patient subcutaneously. In addition, 100 mg prednisone, an anti-inflammatory corticosteroid, is administered PO on days 3-7. This cycle of chemotherapy followed by Maxy-G and prednisone is repeated every 21 days for three to five cycles.

### Example 8

### Same-Day Administration of Chemotherapy and Maxy-G in the Treatment of Hodgkin's Disease

### Doxorubicin, Bleomycin, Vinblastine and Dacarbazine (ABVD Regimen):

A patient with Hodgkin's Disease is treated with a combination chemotherapy regimen of doxorubicin, bleomycin, vinblastine and dacarbazine (“ABVD regimen”). Multi-PEGylated G-CSF variant Maxy-G is administered the same day as the completion of the administration of the chemotherapeutic agents.

The patient is administered a cycle of chemotherapy in which 25 mg/m² doxorubicin, 10 U/m² bleomycin, 6 mg/m² vinblastine, and 375 mg/m² dacarbazine is administered intravenously on day 1 and on day 15, according to standard clinical practice. The exact dosage of each drug depends on a number of factors, such as the weight, age and the severity of the disease, and may be ascertained by those of skill in the art. Within about two to six hours after the completion of administration of the chemotherapeutic agents on day 1 and day 15, 3-12 mg (or alternatively, 50 μg/kg to 150 μg/kg) of Maxy-G is administered to the patient subcutaneously. This cycle of chemotherapy followed by Maxy-G is repeated every 28 days for three to five cycles.

### Example 9

### Same-Day Administration of Chemotherapy and Maxy-G in the Treatment of Ovarian Cancer

### Carboplatin Plus Paclitaxel:

A patient with ovarian cancer is treated with a combination chemotherapy regimen of carboplatin plus paclitaxel. Multi-PEGylated G-CSF variant Maxy-G is administered the same day as the completion of chemotherapy in each cycle.

The patient is administered a cycle of chemotherapy in which about 175 mg/m² paclitaxel is administered intravenously on day 1 over a period of about three hours, followed by carboplatin which is administered intravenously on day 1 to an AUC of 5-6, according to standard clinical practice. The exact dosage of each drug depends on a number of factors, such as the weight, age and the severity of the disease, and may be ascertained by those of skill in the art. Within about two to six hours after completion of the administration of chemotherapy, 3-12 mg (or alternatively, 50 μg/kg to 150 μg/kg) of Maxy-G is administered to the patient subcutaneously. This cycle of chemotherapy followed by Maxy-G is repeated every 21 days for six cycles.

### Topotecan:

A patient with ovarian cancer is treated with a single-agent topotecan regimen. Multi-PEGylated G-CSF variant Maxy-G is administered the same day as the completion of chemotherapy in each cycle.

The patient is administered a cycle of chemotherapy in which about 1.5 mg/m² topotecan is administered intravenously each day for five days, not to exceed 7.5 mg/m² total dose per cycle. The exact dosage of the drug depends on a number of factors, such as the weight, age and the severity of the disease, and may be ascertained by those of skill in the art. Within about two to six hours after completion of administration of the topotecan on day 5, 3-12 mg (or alternatively, 50 μg/kg to 150 μg/kg) of Maxy-G is administered to the patient subcutaneously. This cycle of chemotherapy followed by Maxy-G is repeated every 21 days for 3-5 cycles.

### Example 10

### Next-Day Administration of Mono-PEGylated hG-CSF (Neulasta®) and a Multi-PEGylated G-CSF Variant

This study was performed in order to compare the pharmacodynamic effects of Maxy-G (30 and 100 μg/kg) with those of Neulasta® (100 μg/kg) in rats rendered neutropenic by treatment with CPA (90 mg/kg). The primary parameters were WBC counts and ANC. Furthermore, the pharmacokinetic profiles of Maxy-G and Neulasta® in neutropenic rats were also determined. The G-CSF variants were administered 24 hours (“next day”) after CPA treatment.

Forty (40) male Sprague-Dawley rats (Taconic A/S, Lille Skensved, Denmark) weighing approximately 190 g were used in the study. The animals were housed in macrolon cages (2 per cage) in an environmentally controlled animal room with lights on from 6 PM to 6 AM, a room temperature of 22±1° C. and a relative humidity of 55±5%. The animals were acclimatized for 14 days prior to start of the experiment. The animals were randomized according to weights, assigned to one of six groups of 6-7 animals, and ear-clipped. The rats were fed a standard laboratory rat chow (Altromin, Gesellschaft für Tierernährung mbH, Lage, Germany) and given ad libitum access to tap water acidified with citric acid (˜15 mM) (pH˜3.5).

The vehicle group was administered a formulation of sodium succinate (10 mM) and mannitol (43 mg/mL, 0.24 M), pH 4.0. Maxy-G (30 or 100 μg/mL) was formulated in sodium acetate (10 mM) containing mannitol (43 mg/mL, 0.24 M), pH 4.0. Neulasta® (pegfilgrastim, Amgen, Thousand Oaks, Calif., USA) was formulated in sodium acetate (10 mM), sorbitol (50 mg/mL, 0.27 M) and Tween 20 (33 μg/mL), pH 4.0.

All animals were weighed immediately prior to intraperitoneal (i.p.) administration of cyclophosphamide, (CPA, Sendoxan®, Baxter Oncology, Halle, Germany, 33.33 mg/mL sterile isotonic saline) 90 mg/kg. Twenty-four hours after CPA-exposure, animals were weighed again and administered either vehicle or G-CSF in a total volume of approximately 700 μl (2.7 mL/kg). Vehicle and G-CSF variants were administered s.c. in the neck region.

Blood samples were collected 24 hours prior to CPA dosing and one hour prior to administration of vehicle and the two G-CSF variants (23 hours after CPA dosing). Blood samples were then taken every 24 hours (48, 72, 96, 120, 144, 168, 192, 216, and 240 hours after CPA injection). At each time point, 4 drops (approximately 160 μL) of blood were collected for hematological parameters from a tail vein with an uncoated needle in an 1-mL MiniCollect tube containing EDTA (Greiner Bio-One, Stonehouse, Glos., UK; catalog No. 450-403). The tubes were stored at 4° C.

The blood that was left in the syringe after removing the pharmacodynamic measurement sample was used for the pharmacokinetic portion of the study. The blood was transferred by pipette to a Thrombin-tube (Microvette 300Z, Sarstedt, Nümbrecht, Germany, catalog No. 20.1308.100) and serum was separated by centrifugation (3 min at 5000 g at 4° C.). The serum was then transferred to an Eppendorf tube and stored at −80° C. until assayed. Immediately after the last blood sample collection, all animals were humanely euthanized using O₂/CO₂.

Pharmacodynamic Determination: The effect of Maxy-G and Neulasta® on white blood cell (WBC) count was determined on an ABX Pentra 120 (Horiba ABX, F-34184 Montpellier Cédex 4, France). The relative neutrophil count was manually performed using blood smears. The absolute neutrophil count (ANC) was calculated from the relative level of neutrophils vs. WBCs.

Pharmacodynamic data analysis: WBC counts and ANC were plotted against time using GraphPad Prism software (Version 4.01). Time of CPA dosing was set to time=0.

Pharmacokinetic determination: ELISA: Concentrations of Maxy-G and Neulasta® were determined in each serum sample by ELISA. The ELISA method is based on a commercial ELISA kit (R&D Systems). Briefly, the method involves 1) capture of Maxy-G via a mouse monoclonal anti-human G-CSF antibody coated on the wells of 96-well plates, 2) detection of captured Maxy-G by a biotinylated polyclonal goat anti-human G-CSF antibody, 3) detection of biotinylated antibody by addition of streptavidin conjugated to horseradish peroxidase (HRP), and 4) measurement of HRP activity by addition of a chemiluminescent HRP substrate.

### Results

Pharmacodynamics: Treatment with CPA resulted in profound and prolonged duration of leukopenia and severe neutropenia in rats. This effect was manifested in the vehicle group within 72 h after treatment with CPA. The leukopenia was maintained for 6.3±1.0 days and the severe neutropenia was maintained for 6.6±1.1 days (Table 4).

While subcutaneous administration of Maxy-G (30 or 100 μg/kg) or Neulasta® (100 μg/kg) 24 hours after treatment with CPA did not fully counteract the development of the CPA-induced leukopenia or severe neutropenia, both drugs significantly reduced the duration of the response (Table 4) leading to significantly shorter time-to-recovery (TTR) as compared to vehicle (Table 5). The duration of leukopenia and the time-to-recovery from leukopenia and severe neutropenia were significantly shorter after Maxy-G (100 μg/kg) treatment than after treatment with Neulasta® (100 μg/kg). This suggests that Maxy-G is more potent than Neulasta® in this rat model of severe neutropenia.

TABLE 4
Duration of leukopenia and severe neutropenia in CPA-treated rats
after next day s.c. administration of vehicle, Maxy-G, or Neulasta ®.

| | | | Duration of severe | |
|---|---|---|---|---|
| | Duration of leukopenia | | neutropenia | |
| | (Days with WBC | | (Days with ANC | |
| Compound | <4.0 × 10⁹/L) | N | <0.5 × 10⁹/L) | N |
| Vehicle | 6.3 ± 1.0 | 7 | 6.6 ± 1.1 | 7 |
| Neulasta | 4.8 ± 0.8* | 6 | 2.4 ± 1.7* | 6 |
| (100 μg/kg) | | | | |
| Maxy-G | 3.3 ± 0.5*^{a} | 7 | 2.4 ± 1.0* | 7 |
| (30 μg/kg) | | | | |
| Maxy-G | 2.7 ± 1.1*^{a} | 7 | 2.0 ± 1.0* | 7 |
| (100 μg/kg) | | | | |

Means ± SD. Statistical difference (P) from vehicle is
*P < 0.05, and from Neulasta ®:
^{a}P < 0.05.

TABLE 5
Time-to-recovery from leukopenia and severe neutropenia of
CPA-treated rats after Next Day s.c. administration of vehicle,
Maxy-G, or Neulasta ®.

| | WBC | | ANC | |
|---|---|---|---|---|
| | Time-to-recovery | | Time-to-recovery | |
| Compound | (Days) | N | (Days) | N |
| Vehicle | 7.7 ± 0.5 | 7 | 8.9 ± 0.4 | 7 |
| Neulasta ® | 8.0 ± 0.0 | 6 | 7.2 ± 1.0 | 6 |
| (100 μg/kg) | | | | |
| Maxy-G | 6.4 ± 0.5*^{a} | 7 | 6.0 ± 1.0* | 7 |
| (30 μg/kg) | | | | |
| Maxy-G | 6.0 ± 0.0*^{a} | 7 | 5.1 ± 1.9*^{a} | 7 |
| (100 μg/kg) | | | | |

Means ± SD. Statistical difference (P) from vehicle is
*P < 0.05, and from Neulasta ®:
^{a}P < 0.05.

Pharmacokinetics: After subcutaneous administrations of Maxy-G (30 and 100 μg/kg) and Neulasta® (100 μg/kg) good systemic exposure and distribution were observed allowing estimates of apparent Tₘₐₓ, mean apparent Cₘₐₓ, and AUC₀₋ₜ to be made (Table 6). The apparent Tₘₐₓ values (Table 6) for both compounds were 24 h, the first sampling point after subcutaneous dosing.

The mean AUC₀₋ₜ values for Maxy-G and Neulasta® at the 100 μg/kg dose level were 19461 ng·h/mL (CV of 8.2%) 9366 ng·h/mL (CV of 15.6%), respectively. For Maxy-G at a dose level of 30 μg/kg, the mean AUC₀₋ₜ value was lower than for the higher dose group, as expected; 4108 ng·h/mL (CV of 7.9%).

The mean apparent Cₘₐₓ values at the 100 μg/kg dose level for Maxy-G and Neulasta® were 350.6 ng/mL (CV of 7.9%) and 211.2 ng/mL (CV of 11.3%), respectively. For Maxy-G at a dose level of 30 μg/kg, the mean apparent Cₘₐₓ value was lower than the higher dose group, as expected; 82.7 ng/mL (CV of 7.1%). Consequently, the mean apparent Cₘₐₓ and mean AUC₀₋ₜ for Maxy-G were 1.7-fold and 2.1-fold higher, respectively, than for Neulasta® at the same dose level (100 μg/kg). The mean apparent Cₘₐₓ and mean AUC₀₋ₜ were 4.2-fold and 4.7-fold higher at the 100 μg/kg dose level for Maxy-G as compared to the 30 μg/kg dose level.

TABLE 6
Pharmacokinetic data following subcutaneous s.c. administration of
Maxy-G or Neulasta ®to severely neutropenic rats.

| | | Apparent | Apparent | | |
|---|---|---|---|---|---|
| | Dose | Tₘₐₓ | Cₘₐₓ | AUC₀₋ₜ | |
| Compound | (μg/kg) | (h) | (ng/mL) | (ng · h/mL) | N |
| Neulasta ® | 100 | 24 | 211.2 ± 23.9 | 9366 ± 1464 | 6 |
| Maxy-G | 30 | 24 | 82.7 ± 5.8 | 4108 ± 323* | 7 |
| Maxy-G | 100 | 24 | 350.6 ± 27.8 | 19461 ± 1600*^{a} | 7 |

Mean ± SD.
— Not calculable.
Statistical difference (P) from Neulasta ®(100 μg/kg) is:
*P < 0.05, and from Maxy-G (30 μg/kg):
^{a}P < 0.05.

Overall, the systemic exposure, on an equivalent dose basis, demonstrated that Maxy-G yielded significantly higher values for both mean apparent Cₘₐₓ and AUC₀₋ₜ. The data also indicated that the systemic levels of Maxy-G declined more slowly and had essentially cleared to baseline by 144 h vs. 120 h post dose for Neulasta®.

### Discussion and Conclusion

In the present study, Maxy-G and Neulasta® were shown to be effective treatment in reversing the severe myelosuppressive effect of CPA since both compounds shortened the duration of CPA-induced leukopenia and severe neutropenia.

The present experiment represents a “next-day-administration” regimen of Maxy-G and Neulasta®. The effect of Maxy-G in this model was more pronounced than that of Neulasta®, since both a low dose (30 μg/kg) and an equivalent dose (100 μg/kg) of Maxy-G shortened the duration of leukopenia, and an equivalent dose (100 μg/kg) of Maxy-G shortened the time-to-recovery from leukopenia significantly more than Neulasta®. In addition, Maxy-G shortened the time-to-recovery from severe neutropenia significantly more than Neulasta® at an equivalent dose (100 μg/kg). Therefore, the present study suggests that Maxy-G is more potent than Neulasta® in counteracting the myelosuppressive effect of CPA in rats by stimulating the formation of neutrophils.

The pharmacokinetic parameters of Maxy-G and Neulasta® (Table 6) illustrate that Maxy-G resided longer in serum, accounting for its improved potency.

The results suggest that
1. Maxy-G shortens the duration of leukopenia significantly more than Neulasta® (by 2.1 days) at an equivalent subcutaneous dose (100 μg/kg);
2. Maxy-G shortens the time-to-recovery from leukopenia and severe neutropenia significantly more (2.0 and 2.1 days, respectively) than Neulasta at an equivalent subcutaneous dose (100 μg/kg);
3. The additional reduction by a lower dose of Maxy-G (30 μg/kg) vs. Neulasta® (100 μg/kg) in time-to-recovery from leukopenia and severe neutropenia is 1.6 days and 1.2 days, respectively, while the reduction in duration of leukopenia is 1.5 days;
4. Maxy-G gives a 2.1-fold higher AUC₀₋ₜ value in serum than Neulasta® at an equivalent subcutaneous dose (100 μg/kg);
5. Maxy-G gives a 1.7-fold higher mean apparent Cₘₐₓ value Neulasta® at an equivalent dose (100 μg/kg);
6. Maxy-G and Neulasta® both display mean apparent Tₘₐₓ at 24 h, when samples are taken every 24 h after subcutaneous administration;
7. The mean apparent t_{1/2} for Maxy-G was 16.0 h and 10.6 h in the 30 and 100 μg/kg dosing groups, respectively;
8. The systemic levels of Maxy-G declined more slowly and had essentially cleared by 144 h vs. 120 h post dose for Neulasta®.

Without being bound by any particular theory, in considering the results from the experiments described in Examples 2 and 10, one possible explanation for the differing clinical outcomes involving administration of Neulasta® versus Maxy-G same-day is based on the need for sufficient active G-CSF to be present at a time following elimination of cytotoxic chemotherapy drug(s). It is assumed that for a period of time following same-day administration of Neulasta®, the positive benefits of the G-CSF, with respect to neutrophil mobilization, will be minimized by the presence of the cytotoxic chemotherapy drug(s).

G-CSF stimulates neutrophil production by binding to cell surface G-CSF receptor, subsequently activating the cellular cascades to stimulate proliferation and differentiation, and promote maturation of progenitor cells in the bone marrow to become circulating functioning neutrophils. Chemotherapy agents can cause myelosuppression by damaging cells in the bone marrow and depleting the precursor of mature blood cells. While the progenitor cells are very sensitive, all blood cells are affected in general by chemotherapy agents. Although Neulasta® possesses a longer half-life relative to Neupogen®, it is conceivable that following sufficient elimination of the cytotoxic chemotherapy drug(s) which then allows efficient neutrophil production, levels of Neulasta®, which are more reduced by that point in time, are insufficient to support equivalent neutrophil recovery relative to next-day administration of the G-CSF.

Due to the longer half-life demonstrated by Maxy-G relative to Neulasta®, it is postulated that Maxy-G could maintain equivalent stimulatory activity when administered either next-day or same-day as suggested by the data from Examples 2 and 10.

### Example 11

### Comparison of Same-Day and Next-Day Administration of Maxy-G34 and Neulasta® in a Neutropenic Rat Model

A study was performed to compare the pharmacological effect of equal doses of Maxy-G34 (200 μg/kg) and Neulasta® (200 μg/kg) in rats rendered severely neutropenic by pretreatment with cyclophosphamide (CPA at 90 mg/kg). The test articles were dosed 30 minutes, 2 or 24 hours after CPA administration. The primary parameters were leukocyte or while blood cell (WBC) counts or absolute neutrophil counts (ANC).

### Materials and Methods

Animals: Thirty-eight (38) male, Sprague Dawley rats weighing approximately 250 g were used in the study.

Chemotherapeutic agent: Cyclophosphamide (CPA, Sendoxan)

Comparator mono-PEGylated G-CSF: Neulasta® (see Example 2) was prepared in 10 mM Na-acetate containing 50 mg/mL sorbitol and 0.033 mg/mL Tween-20 at concentrations of 0.2 mg/mL Neulasta®.

Test multi-PEGylated G-CSF variant: “Maxy-G” (see Example 2) was prepared in a vehicle solution of 10 mM Na-Acetate containing 45 mg/mL mannitol and 0.05 mg/mL Tween-20 (pH=4.0) at concentrations of 0.2 mg/mL Maxy-G34.

Vehicle: Aqueous solution of 10 mM Na-Acetate containing 45 mg/mL mannitol and 0.05 mg/mL Tween-20 (pH 4.0).

Study Design:

| | | Time of | | Dose level | Dose volume | Dose conc. | Number |
|---|---|---|---|---|---|---|---|
| Group | Treatment | Treatment | | (mg/kg) | (mL/kg) | (mg/mL) | in group |
| 1. | vehicle | ½ | hr after CPA | 0 | 1 | 0 | 5 |
| 2. | Maxy-G | ½ | hr after CPA | 0.2 | 1 | 0.2 | 6 |
| 3. | Maxy-G | ½ | hr after CPA | 0.2 | 1 | 0.2 | 6 |
| 4. | Maxy-G | 24 | hr after CPA | 0.2 | 1 | 0.2 | 6 |
| 5. | Neulasta ® | ½ | hr after CPA | 0.2 | 1 | 0.2 | 5 |
| 6. | Neulasta ® | 2 | hr after CPA | 0.2 | 1 | 0.2 | 5 |
| 7. | Neulasta ® | 24 | hr after CPA | 0.2 | 1 | 0.2 | 5 |

Dosing and Blood Collection Procedures: One day prior to the start of the study (Day −1), the animals were assigned to one of seven groups (vehicle at ½ hour after CPA; or 200 μg/kg Maxy-G at ½ hour, 2 hours or 24 hours after CPA; or 200 μg/kg Neulasta® at ½ hour, 2 hours or 24 hours after CPA) and a tail vein blood sample was collected from each animal for use as a baseline determination. The start of the study was designated Day 0. On Day 0, the animals were individually weighed and were administered CPA intraperitoneally (i.p.) at a dose of 90 mg/kg to induce neutropenia. At ½ hour, 2 hours or 24 hours after the CPA administration, Maxy-G or Neulasta® was administered subcutaneously in the scruff of the neck at 1 mL/kg body weight. For the vehicle group, vehicle was injected ½ hour after the CPA administration. Blood sampling from tail vein for pharmacology analysis started approximately 24 hours (Day 1) after the injection of CPA and continued for a total of 9 days. The final blood sample was obtained on Day 9.

Pharmacodynamic Determinations: The effect of Maxy-G and Neulasta® on leukocyte or white blood cell count (WBC) and other hematological parameters was determined. The relative neutrophil count was manually performed using blood smears. The absolute neutrophil count (ANC) was manually calculated from the relative level of ANC vs. WBC.

### Results

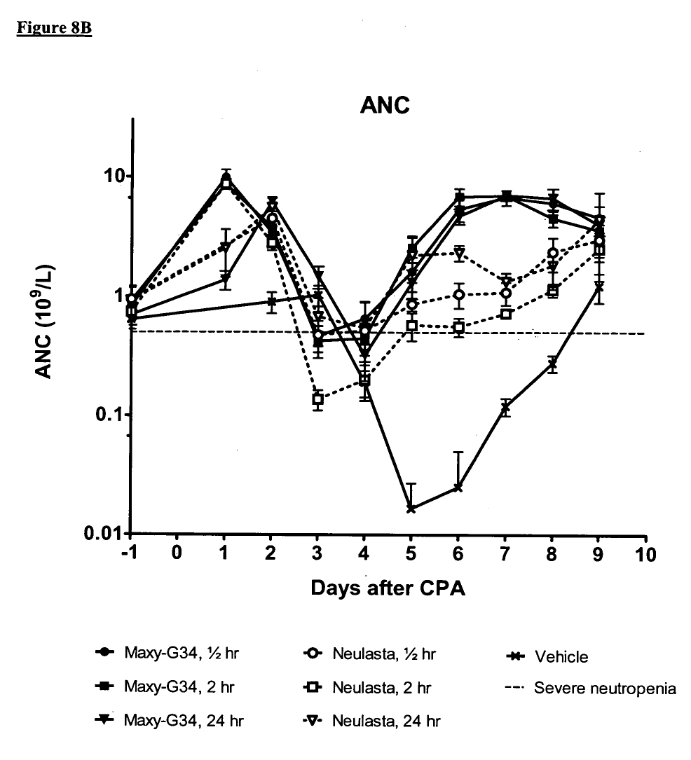

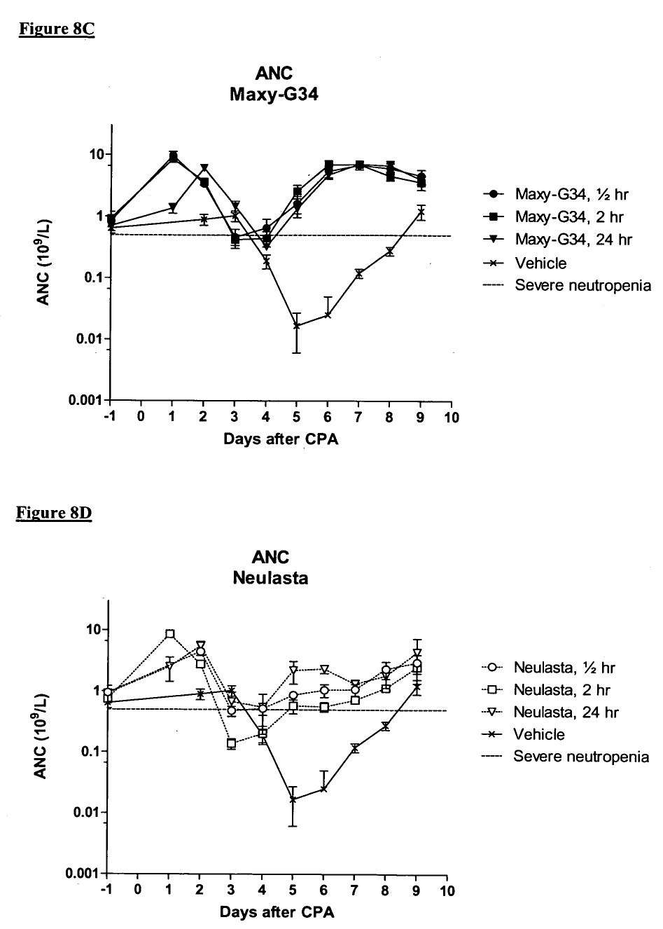

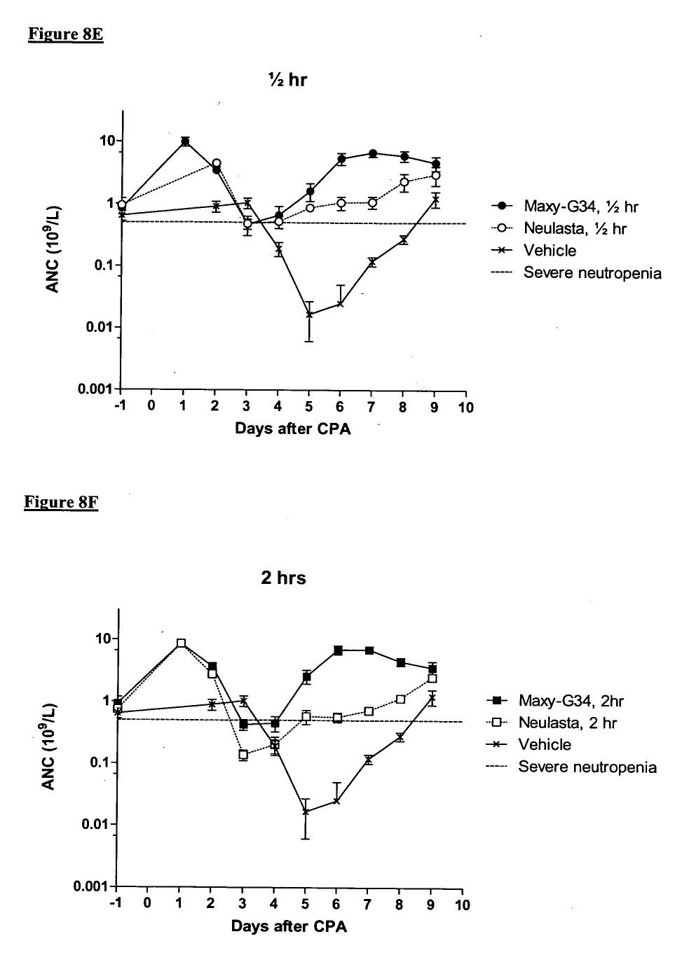

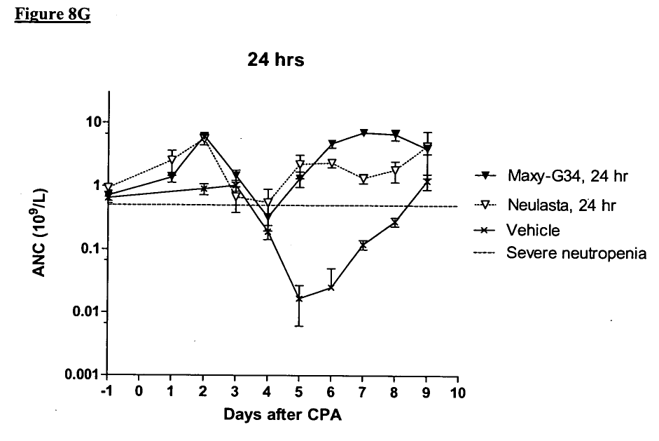

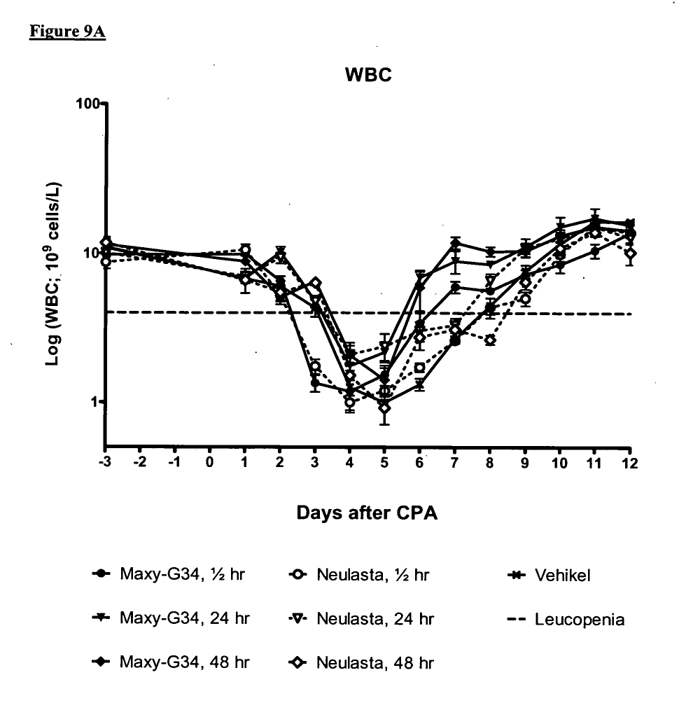

The effects of Maxy-G and Neulasta® on WBC counts in rats rendered neutropenic by CPA are shown in FIG. 8A. Treatment with 200 μg/kg Maxy-G substantially reduced leukopenia compared to treatment with vehicle. The decrease of WBC counts was less severe and the recovery back to baseline levels was more rapid in all three Maxy-G treated groups compared to vehicle-treated group. The timing of administration of Maxy-G34 did not substantially alter its effect in reducing leukopenia. Neulasta at 200 μg/kg also showed improvement in leukopenia as compared to vehicle but is less effective than Maxy-G especially when it was administered 2 hours after CPA.

The effects of Maxy-G and Neulasta® on ANC in rats rendered neutropenic by CPA are shown in FIGS. 8B to 8G. The overall results of ANC changes are consistent with the results of WBC changes. CPA caused severe neutropenia as expected. Treatment with 200 μg/kg Maxy-G substantially reduced neutropenia as compared to treatment with vehicle. The effects of Maxy-G on dampening the reduction of ANC and hastening the recovery of ANC to baseline level were evident in all Maxy-G-treated groups and the Maxy-G was similarly effective regardless of the timing of administration post-CPA. Neulasta® was also effective compared to vehicle albeit the rate of ANC recovery was slower than Maxy-G. Neulasta® was affected by the timing of administration post CPA and was less effective when administered 2 hours post-CPA than ½ hour or 24 hours.

### Conclusion

These data showed that Maxy-G34 was effective in reducing neutropenia similarly when administered at ½ hour, 2 hours or 24 hours post-CPA. The effect of Neulasta® was comparable with Maxy-G when administered 24 hours post-CPA but it was less effective than Maxy-G when administered ½ hour or 2 hours post-CPA.

### Example 12

### Comparison of Same-Day, Next-Day and Two-Day Administration of Maxy-G34 and Neulasta® in Neutropenic Rat

A study was performed to compare the pharmacological effect of equal doses of Maxy-G34 (100 μg/kg) and Neulasta® (100 μg/kg) in rats rendered severely neutropenic by pretreatment with cyclophosphamide (CPA at 90 mg/kg). The test articles were dosed 30 minutes, 24 or 48 hours after CPA administration. The primary parameters were leukocyte or white blood cell (WBC) counts or absolute neutrophil counts (ANC).

### Materials and Methods

Animals: Fifty (50) male, Sprague Dawley rats weighing approximately 250 g were used in the study.

Chemotherapeutic agent: Cyclophosphamide (CPA, Sendoxan)

Comparator mono-PEGylated G-CSF: Neulasta® (see Example 2) was prepared in 10 mM Na-acetate containing 50 mg/mL sorbitol and 0.033 mg/mL Tween-20 at concentrations of 0.2 mg/mL Neulasta®.

Test multi-PEGylated G-CSF variant: “Maxy-G” (see Example 2) was prepared in a vehicle solution of 10 mM Na-Acetate containing 45 mg/mL mannitol and 0.05 mg/mL Tween-20 (pH=4.0) at concentrations of 0.2 mg/mL Maxy-G34.

Vehicle: Aqueous solution of 10 mM Na-Acetate containing 45 mg/mL mannitol and 0.05 mg/mL Tween-20 (pH 4.0).

Study Design:

| | | Time of | | Dose level | Dose volume | Dose conc. | Number |
|---|---|---|---|---|---|---|---|
| Group | Treatment | Treatment | | (mg/kg) | (mL/kg) | (mg/mL) | in group |
| 1. | vehicle | ½ | hr after CPA | 0 | 1 | 0 | 8 |
| 2. | Maxy-G | ½ | hr after CPA | 0.1 | 1 | 0.1 | 8 |
| 3. | Maxy-G | 24 | hr after CPA | 0.1 | 1 | 0.1 | 8 |
| 4. | Maxy-G | 48 | hr after CPA | 0.1 | 1 | 0.1 | 5 |
| 5. | Neulasta ® | ½ | hr after CPA | 0.1 | 1 | 0.1 | 8 |
| 6. | Neulasta ® | 24 | hr after CPA | 0.1 | 1 | 0.1 | 8 |
| 7. | Neulasta ® | 48 | hr after CPA | 0.1 | 1 | 0.1 | 5 |

Dosing and Blood Collection Procedures: Three days before to the start of the study (Day −3), the animals were assigned to one of seven groups (vehicle at ½ hour after CPA; or 100 μg/kg Maxy-G at ½ hour, 24 hours or 48 hours after CPA; or 100 μg/kg Neulasta® at ½ hour, 24 hours or 48 hours after CPA) and a tail vein blood sample was collected from each animal for use as a baseline determination. The start of the study was designated Day 0. On Day 0, the animals were individually weighed and were administered CPA intraperitoneally (i.p.) at a dose of 90 mg/kg to induce neutropenia. At ½ hour, 24 hours or 48 hours after the CPA administration, Maxy-G or Neulasta® was administered subcutaneously in the scruff of the neck at 1 mL/kg body weight. For the vehicle group, vehicle was injected ½ hour after the CPA administration. Blood sampling from tail vein for pharmacology analysis started approximately 24 hours (Day 1) after the injection of CPA and continued for a total of 12 days. The final blood sample was obtained on Day 12.

Pharmacodynamic Determinations: The effect of Maxy-G and Neulasta® on leukocyte or white blood cell count (WBC) and other hematological parameters was determined. The relative neutrophil count was manually performed using blood smears. The absolute neutrophil count (ANC) was manually calculated from the relative level of ANC vs. WBC.

### Results

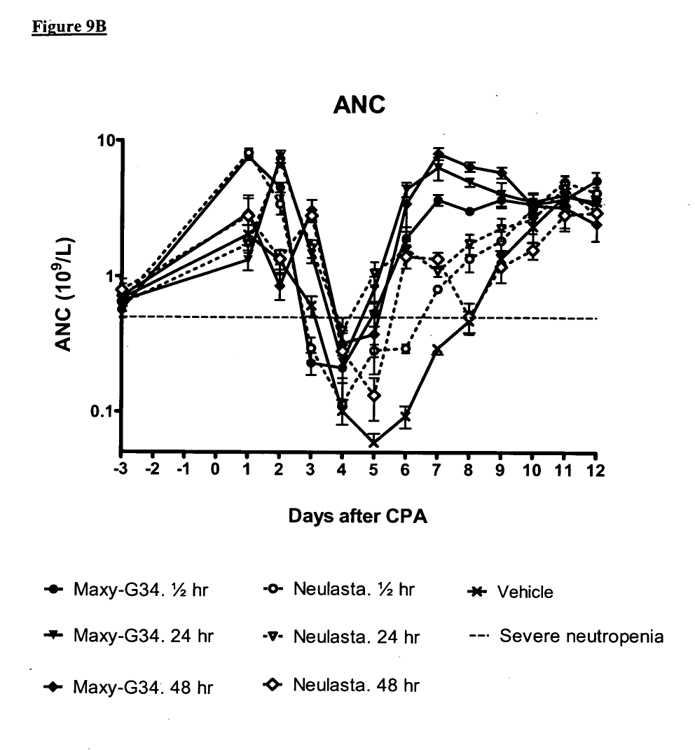

The effects of Maxy-G and Neulasta® on WBC counts in rats rendered neutropenic by CPA are shown in FIG. 9A and the effects on ANC are shown in FIG. 9B.

TABLE 7
Comparison of the effects of Maxy-G to Neulasta ® in the
recovery and duration of leukopenia in CPA-induced rats

| | | WBC | WBC |
|---|---|---|---|
| | | Time to Recovery | Duration of Leukopenia |
| Treatment Group | N | (Days*) | (Days*) |
| Vehicle | 8 | 8.5 ± 0.5 | 5.1 ± 1.4 |
| Maxy-G34, ½ hr | 8 | 6.8 ± 0.4 | 3.8 ± 0.5 |
| Neulasta, ½ hr | 8 | 8.8 ± 0.8 | 6.0 ± 0.9 |
| Maxy-G34, 24 hr | 8 | 6.3 ± 0.8 | 2.4 ± 0.7 |
| Neulasta, 24 hr | 8 | 7.9 ± 1.5 | 4.3 ± 1.5 |
| Maxy-G34, 48 hr | 5 | 6.6 ± 0.5 | 2.8 ± 0.8 |
| Neulasta, 48 hr | 5 | 9.0 ± 0.0 | 5.2 ± 0.5 |

*Data are represented as mean ± standard deviation.

Treatment with 100 μg/kg Maxy-G shortened the time to recovery and the duration of leukopenia compared to treatment with vehicle and Neulasta®. Table 7 shows that Maxy-G was effective when administered at ½ hour, 24 hours or 48 hours after CPA. The effect of Maxy-G on shortening the time to recovery of leukopenia was similar when administered at ½, 24 or 48 hours post CPA. Maxy-G may be slightly less effective in shortening the duration of leukopenia when administered at ½ hour relative to 24 or 48 hours post CPA. Neulasta® showed minimal to no effect on leukopenia compared to vehicle when administered ½, 24 or 48 hours post-CPA.

TABLE 8
Comparison of the effects of Maxy-G to Neulasta ® in the
recovery and duration of severe neutropenia in CPA-induced rats

| | | ANC | ANC |
|---|---|---|---|
| | | Time to Recovery | Duration of Neutropenia |
| Treatment Group | N | (Days*) | (Days*) |
| Vehicle | 8 | 8.5 ± 0.5 | 5.3 ± 0.9 |
| Maxy-G34, ½ hr | 8 | 5.6 ± 1.0 | 2.9 ± 0.6 |
| Neulasta, ½ hr | 8 | 7.0 ± 0.0 | 4.1 ± 0.6 |
| Maxy-G34, 24 hr | 8 | 4.9 ± 1.8 | 1.8 ± 1.2 |
| Neulasta, 24 hr | 8 | 4.0 ± 1.6 | 1.1 ± 1.0 |
| Maxy-G34, 48 hr | 5 | 5.6 ± 0.5 | 2.0 ± 0.7 |
| Neulasta, 48 hr | 5 | 6.0 ± 0.0 | 2.2 ± 0.8 |

*Data are represented as mean ± standard deviation.

Treatment with 100 μg/kg Maxy-G shortened the time to recovery and the duration of severe neutropenia induced by CPA compared to treatment with vehicle. Table 8 shows that Maxy-G was effective when administered at ½ hour, 24 hours or 48 hours after CPA and that the shortening of time to recovery and duration of severe neutropenia were similarly effected when Maxy-G was administered at ½ hour, 24 hours or 48 hours post CPA. Neulasta® showed comparable effectiveness compared to Maxy-G when administered 24 or 48 hours after CPA. Neulasta® was less effective than Maxy-G when administered ½ hour post-CPA in shortening the duration of severe neutropenia.

### Conclusion

These data shows that Maxy-G34 was effective in reducing neutropenia when administered from ½ hour to 48 hours post-CPA. The effectiveness of Neulasta® was comparable with Maxy-G when administered 24 hours or 48 hours post-CPA but less effective than Maxy-G when administered ½ hour post-CPA.

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

We claim:

1. A method for treating or preventing neutropenia in a patient receiving chemotherapy, comprising administering to said patient a multi-PEGylated G-CSF variant in an amount effective to reduce chemotherapy-induced neutropenia, wherein the multi-PEGylated G-CSF variant is administered to the patient on the same day as chemotherapy.

2. The method of claim 1, wherein the multi-PEGylated G-CSF variant is administered to the patient within about 6 hours of the completion of chemotherapy.

3. The method of claim 1, wherein the multi-PEGylated G-CSF variant is administered to the patient within about 5 hours of the completion of chemotherapy.

4. The method of claim 1, wherein the multi-PEGylated G-CSF variant is administered to the patient within about 4 hours of the completion of chemotherapy.

5. The method of claim 1, wherein the multi-PEGylated G-CSF variant is administered to the patient within about 2 hours of the completion of chemotherapy.

6. The method of claim 1, wherein the multi-PEGylated G-CSF variant is administered to the patient within about one-half hour of the completion of chemotherapy.

7. The method of claim 1, wherein the multi-PEGylated G-CSF variant comprises the amino acid sequence of SEQ ID NO: 1 and at least one substitution relative to SEQ ID NO: 1 selected from the group consisting of T1K, P2K, L3K, G4K, P5K, A6K, S7K, S8K, L9K, P10K, Q11K, S12K, F13K, L14K, L15K, E19K, Q20K, V21K, Q25K, G26K, D27K, A29K, A30K, E33K, A37K, T38K, Y39K, L41K, H43K, P44K, E45K, E46K, V48K, L49K, L50K, H52K, S53K, L54K, I56K, P57K, P60K, L61K, S62K, S63K, P65K, S66K, Q67K, A68K, L69K, Q70K, L71K, A72K, G73K, S76K, Q77K, L78K, S80K, F83K, Q86K, G87K, Q90K, E93K, G94K, S96K, P97K, E98K, L99K, G100K, P101K, T102K, D104K, T105K, Q107K, L108K, D109K, A111K, D112K, F113K, T115K, T116K, W118K, Q119K, Q120K, M121K, E122K, E123K, L124K, M126K, A127K, P128K, A129K, L130K, Q131K, P132K, T133K, Q134K, G135K, A136K, M137K, P138K, A139K, A141K, S142K, A143K, F144K, Q145K, S155K, H156K, Q158K, S159K, L161K, E162K, V163K, S164K, Y165K, V167K, L168K, H170K, L171K, A172K, Q173K and P174K.

8. The method of claim 7, wherein the multi-PEGylated G-CSF variant comprises at least one substitution selected from the group consisting of Q70K, Q90K, T105K Q120K, T133K, S159K and H170K.

9. The method of claim 1, wherein the multi-PEGylated G-CSF variant comprises the amino acid sequence of SEQ ID NO: 1 with the substitutions K16R, K34R, K40R, T105K and S159K.

10. The method of claim 1, wherein the multi-PEGylated G-CSF variant comprises the amino acid sequence of SEQ ID NO: 1 with at least one substitution selected from the group consisting of K16R, K16Q, K34R, K34Q, K40R and K40Q.

11. The method of claim 1, wherein the multi-PEGylated G-CSF variant comprises 2-6 polyethylene glycol (PEG) moieties each with a molecular weight of about 1-10 kDa.

12. The method of claim 11, wherein the multi-PEGylated G-CSF variant comprises a PEG moiety attached to the N-terminus and a PEG moiety attached to a lysine residue.

13. The method claim 11, wherein the multi-PEGylated G-CSF variant comprises 2-4, 3-5, 4-6, 2-3, 3-4, 4-5 or 5-6 attached PEG moieties.

14. The method of claim 13, wherein the PEGylated G-CSF comprises 2-4 polyethylene glycol moieties with a molecular weight of about 4-6 kDa.

15. The method of claim 1, wherein the multi-PEGylated G-CSF variant comprises the amino acid sequence of SEQ ID NO: 1 with one or more substitution selected from K16R, K16Q, K34R, K34Q, K40R and K40Q, and one or more substitution selected from Q70K, Q90K, T105K, Q120K, T133K, and S159K, and comprises 2-6 attached PEG moieties each with a molecular weight of about 1-10 kDa.

16. The method of claim 15, wherein the multi-PEGylated G-CSF variant comprises one or more substitution selected from K16R, K34R, and K40R, and at least one substitution selected from T105K and S159K, and comprises 2-4 attached PEG moieties each with a molecular weight of about 1-10 kDa.

17. The method of claim 16, wherein the multi-PEGylated G-CSF variant comprises the substitutions K16R, K34R, K40R, T105K and S159K, and comprises 2-4 attached PEG moieties each with a molecular weight of about 4-6 kDa.

18. The method of claim 9, wherein the G-CSF variant component of the multi-PEGylated G-CSF variant consists of the amino acid sequence of SEQ ID NO: 1 with the substitutions K16R, K34R, K40R, T105K and S159K.

19. The method of claim 18, wherein the multi-PEGylated G-CSF variant is a mixture of positional PEG isomer species.

20. The method of claim 19, wherein the mixture of positional PEG isomer species comprises at least 2 species of positional PEG isomers each having 3 attached PEG moieties, wherein one of the isomers has PEG moieties attached at the N-terminal, Lys23 and Lys159, and the other isomer has PEG moieties attached at the N-terminal, Lys105 and Lys159.

21. The method of claim 20, wherein the PEG moieties each have a molecular weight of about 1-10 kDa.

22. The method of claim 21, wherein the PEG moieties each have a molecular weight of about 5 kDa.

23. The method of claim 1, wherein the multi-PEGylated G-CSF variant exhibits an improved pharmacokinetic property compared to Neulasta® (pegfilgrastim) when tested under comparable conditions in an animal model.

24. The method of claim 18, wherein the multi-PEGylated G-CSF variant exhibits an increased serum half-life compared to Neulasta® in a rat animal model.

25. The method of claim 18, wherein the multi-PEGylated G-CSF variant exhibits an increased AUC compared to Neulasta® in a rat animal model.

26. The method of claim 1, wherein the multi-PEGylated G-CSF variant is administered in a dose of from about 2-15 mg per patient.

27. The method of claim 1, wherein the multi-PEGylated G-CSF variant is administered in a dose of from about 1-5 mg per patient.

28. The method of claim 1, wherein the chemotherapy comprises administration of a chemotherapeutic agent selected from the group consisting of alkylating agents, plant alkaloids, antitumor antibiotics, antimetabolites and topoisomerase inhibitors.

29. The method of claim 28, wherein the chemotherapeutic agent is an alkylating agent selected from the group consisting of: mustard gas derivatives, such as Cyclophosphamide, Chlorambucil, Ifosfamide, Mechlorethamine or Melphalan; ethylenimines, such as Hexamethylmelamine or Thiotepa; alkylsulfonates such as Busulfan; hydrazines and triazines such as Altretamine, Dacarbazine, Procarbazine or Temozolomide; nitrosureas such as Carmustine, Lomustine or Streptozocin; and inorganic metal complex agents such as Cisplatin, Carboplatin or Oxaliplatin.

30. The method of claim 28, wherein the chemotherapeutic agent is a plant alkaloid selected from the group consisting of taxanes (e.g., Docetaxel or Paclitaxel), vinca alkaloids (e.g., Vinblastine, Vincristine or Vinorelbine), camptothecan analogs (e.g., Irinotecan or Topotecan) and podophyllotoxins (e.g., Etoposide or Tenisopide).

31. The method of claim 28, wherein the chemotherapeutic agent is an antitumor antibiotic selected from the group consisting of: anthracyclines, such as Daunorubicin, Doxorubicin, Epirubicin, Idarubicin, or Mitoxantrone; chromomycins, such as Dactinomycin or Plicamycin; and other antitumor antibiotics such as Bleomycin or Mitomycin.

32. The method of claim 28, wherein the chemotherapeutic agent is an antimetabolite selected from the group consisting of: folic acid antagonists, such as Methotrexate; pyrimidine antagonists, such as Capecitabine, Cytarabine, 5-Fluorouracil (5-FU), Foxuridine or Gemcitabine; purine antagonists, such as 6-Mercaptopurine or 6-Thioguanine; adenosine deaminase inhibitors, such as Cladribine, Fludarabine, Nelarabine or Pentostatin; and ribonucleotide reductase inhibitors, such as Hydroxyurea.

33. The method of claim 28, wherein the chemotherapeutic agent is a topoisomerase inhibitor selected from the group consisting of: topoisomerase I inhibitors, such as Ironotecan or Topotecan; and topoisomerase II inhibitors, such as Amsacrine, Etoposide, Etoposide Phosphate or Teniposide.

34. The method of claim 28, wherein the chemotherapeutic agent is selected from the group consisting of Carboplatin, Doxorubicin, Cyclophosphamide, Paclitaxel (Taxol) and Vincristine.

35. The method of claim 1, wherein the chemotherapy is for treatment of a cancer selected from breast cancer, non-small cell lung cancer, small cell lung cancer, colorectal cancer, uterine cancer, ovarian cancer, non-Hodgkin's lymphoma (NHL), and Hodgkin's disease.

36. The method of claim 1, wherein said patient is receiving multiple cycles of chemotherapy, and wherein in each cycle of chemotherapy, the multi-PEGylated G-CSF variant is administered to the patient on the same day as chemotherapy.
